SEQUENCE LISTING (29 sequences)

SEQ ID NO: 1 - protein, 418 aa, homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 359 aa, homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKVRSP

SEQ ID NO: 3 - protein, 306 aa, homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRR

SEQ ID NO: 4 - protein, 4 aa, artificial sequence
  peptide fragment
VRSP

SEQ ID NO: 5 - protein, 18 aa, artificial sequence
  mass spectrometry peptide
LYHSEAFTVNFGDTEEAK

SEQ ID NO: 6 - protein, 15 aa, artificial sequence
  mass spectrometry peptide
LQHLENELTHDIITK

SEQ ID NO: 7 - protein, 15 aa, artificial sequence
  mass spectrometry peptide
TDTSHHDQDHPTFNK

SEQ ID NO: 8 - protein, 19 aa, artificial sequence
  mass spectrometry peptide
LYHSEAFTVNFGDTEEAKK

SEQ ID NO: 9 - protein, 15 aa, artificial sequence
  mass spectrometry peptide
TDTSHHDQDHPTFNK

SEQ ID NO: 10 - protein, 17 aa, artificial sequence
  mass spectrometry peptide
QPFVQGLALYTPVVLPR

SEQ ID NO: 11 - protein, 17 aa, artificial sequence
  mass spectrometry peptide
VLSALQAVQGLLVAQGR

SEQ ID NO: 12 - protein, 14 aa, artificial sequence
  mass spectrometry peptide
EHAVEGDCDFQLLK

SEQ ID NO: 13 - protein, 20 aa, artificial sequence
  mass spectrometry peptide
HTFMGVVSLGSPSGEVSHPR

SEQ ID NO: 14 - protein, 20 aa, artificial sequence
  mass spectrometry peptide
HTFMGVVSLGSPSGEVSHPR

SEQ ID NO: 15 - protein, 13 aa, artificial sequence
  mass spectrometry peptide
GAYPLSIEPIGVR

SEQ ID NO: 16 - protein, 14 aa, artificial sequence
  mass spectrometry peptide
DLYSGLIGPLIVCR

SEQ ID NO: 17 - protein, 12 aa, artificial sequence
  mass spectrometry peptide
TSLEDFYLDEER

SEQ ID NO: 18 - protein, 14 aa, artificial sequence
  mass spectrometry peptide
LAAAVSNFGYDLYR

SEQ ID NO: 19 - protein, 14 aa, artificial sequence
  mass spectrometry peptide
YVMLPVADQDQCIR

SEQ ID NO: 20 - protein, 9 aa, artificial sequence
  mass spectrometry peptide
VGYVSGWGR

SEQ ID NO: 21 - protein, 11 aa, artificial sequence
  mass spectrometry peptide
AREDIFMETLK

SEQ ID NO: 22 - protein, 12 aa, artificial sequence
  mass spectrometry peptide
AYLEEECPATLR

SEQ ID NO: 23 - protein, 12 aa, artificial sequence
  mass spectrometry peptide
YSLTYIYTGLSK

SEQ ID NO: 24 - protein, 485 aa, homo sapiens
MRKRAPQSEMAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 25 - protein, 367 aa, homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCTVFQTQPVTSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 26 - protein, 1065 aa, homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 27 - protein, 418 aa, homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 28 - protein, 406 aa, homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 29 - protein, 298 aa, homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

(19) United States
(12) Patent Application Publication
(10) Pub. No.: US 2014/0235473 A1
(43) Pub. Date: Aug. 21, 2014

(54) Diagnosis and/or Prognosis of Parkinson's Disease Dementia

(71) Applicants: Markus Otto, Ulm (DE); Stefan Lehnert, Blaustein (DE); Sarah Jesse, Bollingen (DE); Olaf Jahn, Gottingen (DE)

(72) Inventors: Markus Otto, Ulm (DE); Stefan Lehnert, Blaustein (DE); Sarah Jesse, Bollingen (DE); Olaf Jahn, Gottingen (DE)

(21) Appl. No.: 14/127,835

(22) PCT Filed: Jun. 22, 2012
(86) PCT No.: PCT/EP2012/062087

§ 371 Date: Apr. 2, 2014

Related U.S. Application Data
(60) Provisional application No. 61/501,019, filed on Jun. 24, 2011.

(51) Int. Cl. G01N 33/68; C07K 16/38; C07K 16/18; C07K 16/44

(52) U.S. Cl. CPC G01N 33/6896; C07K 16/44; C07K 16/38; C07K 16/18
USPC 506/9; 435/7.92; 530/387.5; 530/387.9; 422/69; 435/7.1; 435/23; 435/287.2; 436/501; 436/87; 506/12; 506/18

(57) ABSTRACT

The present Invention provides a method for diagnosing and/or prognosing Parkinson's disease dementia (PDD) comprising the step of detecting O-glycosylation. in a protein comprising a Ser/Thr motive, in particular Serpin A1, and/or the level of sialic acid on a protein comprising a Ser/Thr motive, in particular Serpin A1. Further, the present invention relates to a molecule for detecting O-linked glycomoieties in a protein comprising a Ser/Thr motive, in particular Serpin A1, and/or glycosylated i so forms of a Ser/Thr motive comprising protein, in particular Serpin A1, for use in the diagnosis and/or prognosis of Parkinson's disease dementia (PDD), Furthermore, the present invention relates to means for diagnosing and/or prognosing Parkinson's disease dementia (PDD) and a kit for diagnosing and/or prognosing Parkinson's disease dementia (PDD).

The present invention provides a method for diagnosing and/or prognosing Parkinson's disease dementia (PDD) comprising the step of detecting O-glycosylation in a protein comprising a Ser/Thr motive, in particular Serpin A1, and/or the level of sialic acid on a protein comprising a Ser/Thr motive, in particular Serpin A1. Further, the present invention relates to a molecule for detecting O-linked glycomoieties in a protein comprising a Ser/Thr motive, in particular Serpin A1, and/or glycosylated isoforms of a Ser/Thr motive comprising protein, in particular Serpin A1, for use in the diagnosis and/or prognosis of Parkinson's disease dementia (PDD). Furthermore, the present invention relates to means for diagnosing and/or prognosing Parkinson's disease dementia (PDD) and a kit for diagnosing and/or prognosing Parkinson's disease dementia (PDD).

### BACKGROUND OF THE INVENTION

Parkinson's disease (hereinafter also referred to as PD) is a degenerative disorder of the central nervous system. It is connected to the death of dopaminergic cells of a region of the mid-brain, the substantia nigra. Initial symptoms include shaking, rigidity, impairment, particularly slowness of movement. Other symptoms include sensory, sleep and behavioural disorders. In some cases, cognitive disorders can appear at a later stage, including dementia.

An increasing prevalence for PD can be detected in advanced age, with 1% among 60-year-olds and 3% in the 80-year-old age-group (Di Napoli, M., I. M. Shah, and D. A. Stewart, Molecular pathways and genetic aspects of Parkinson's disease: from bench to bedside. Expert Rev Neurother, 2007. 7(12): p. 1693-729). Of note is that patients with PD have a roughly 6-times higher risk to develop a dementia than an age-matched healthy control group (Rongve, A. A., D., Management of Parkinson's disease dementia: practical considerations. Drugs Aging, 2006. 23(10): p. 807-822). Up to 50% of the patients show cognitive decline in terms of a mild cognitive impairment already in early stages (Caballol, N., M. J. Marti, and E. Tolosa, Cognitive dysfunction and dementia in Parkinson disease. Mov Disord, 2007. 22 Suppl 17: p. S358-66) whereby 30% of these patients develop a dementive syndrome in the course of the disease which often also includes changes in personality (Dubois, B. and B. Pillon, Cognitive deficits in Parkinson's disease. J Neurol, 1997. 244(1): p. 2-8). The dementive syndrome usually develops after approximately 8 to 10 years and has a huge influence on the course of the disease and also on the social environment with higher requirements for families and caretakers during everyday care which put a psychological strain on the patient and the environment (Aarsland, D., et al., Mental symptoms in Parkinson's disease are important contributors to caregiver distress. Int J Geriatr Psychiatry, 1999. 14(10): p. 866-74), leading to increased stress during home care (Caballol, N., M. J. Marti, and E. Tolosa, Cognitive dysfunction and dementia in Parkinson disease. Mov Disord, 2007. 22 Suppl 17: p. S358-66) with growing need for professional care. The dementive syndrome also goes along with a worsening prognosis with respect to disease-progression and expectancy of life for the patients (Louis, E. D., et al., Mortality from Parkinson disease. Arch Neurol, 1997. 54(3): p. 260-4). However, early treatment is critical since according to present knowledge early therapy of cognitive deficits is crucial to its success (Singh, B. and J. T. O'Brien, When should drug treatment be started for people with dementia? Maturitas, 2009. 62(3): p. 230-4). Therefore, there is a clear need for an early marker to define patients at risk.

Neuropathologically, PD dementia (hereinafter also referred to as PDD) is characterized by the occurrence of cortical Lewy bodies that do also occur in patients with Lewy-body-dementia, another clinical entity of dementia with a more rapid progression (Goedert, M. and MG. Spillantini, Lewy body diseases and multiple system atrophy as alpha-synucleinopathies. Mol Psychiatry, 1998. 3(6): p. 462-5; Jellinger, K. A., A critical evaluation of current staging of alpha-synuclein pathology in Lewy body disorders. Biochim Biophys Acta, 2009. 1792(7): p. 730-40; Jellinger, K. A. and J. Atterns, Prevalence and impact of vascular and Alzheimer pathologies in Lewy body disease. Acta Neuropathol, 2008. 115(4): p. 427-36; Mukaetova-Ladinska, E. B. and I. G. McKeith, Pathophysiology of synuclein aggregation in Lewy body disease. Mech Ageing Dev, 2006. 127(2): p. 188-202). It has been shown that these Lewy bodies contain a-synuclein, a presynaptic filament protein that is expressed at high concentrations in the terminal endings of neurons. Therefore, an obvious working theory is that these Lewy bodies are directly linked to the neuropathological processes, especially that a-synuclein inclusions are mostly present in surviving cells and less so in apoptotic cells, suggesting that these inclusions may play a protective role in cell death by sequestering toxic molecular species (Tanaka, M., et al., Aggresomes formed by alpha-synuclein and synphilin-1 are cytoprotective. J Biol Chem, 2004. 279(6): p. 4625-31; Kramer, M. L. and W. J. Schulz-Schaeffer, Presynaptic alpha-synuclein aggregates, not Lewy bodies, cause neurodegeneration in dementia with Lewy bodies. J Neurosci, 2007. 27(6): p. 1405-10). Regarding the formation of a-synuclein containing inclusion bodies and their importance in neuropathological alterations, Braak et al. were able to indicate a topographical extent of these lesions with an initial onset in the dorsal motor nucleus of the glossopharyngeal as well as vagal nerves and anterior olfactory nucleus in the brain stem, proceeding with an ascending course to cortical structures, beginning with the anteromedial temporal mesocortex (Braak, H. and E. Braak, Pathoanatomy of Parkinson's disease. J Neurol, 2000. 247 Suppl 2: p. II3-10; Braak, H., et al., Parkinson's disease: affection of brain stein nuclei controlling premotor and motor neurons of the somatomotor system. Acta Neuropathol, 2000. 99(5): p. 489-95; Wolters, E. and H. Braak, Parkinson's disease: premotor clinico-pathological correlations. J Neural Transco Suppl, 2006(70): p. 309-19). As a possible link between neurotoxicity, aggregation and propagation it might be concluded that species of neurotoxic oligomers can be transformed to oligomers which are not neurotoxic, but have a higher tendency of further aggregation (Danzer, K. M., et al., Different species of alpha-synuclein oligomers induce calcium influx and seeding. J Neurosci, 2007. 27(34): p. 9220-32; Schnack, C., et al., Protein array analysis of oligomerization-induced changes in alpha-synuclein protein-protein interactions points to an interference with Cdc42 effector proteins. Neuroscience, 2008. 154(4): p. 1450-7).

The present inventors and others made attempts to improve the early diagnosis of PDD in PD patients by measurement of a-synuclein or proposed a-synuclein aggregates and by known biomarkers in cerebrospinal fluid and serum (Jesse, S. S., P.; Lehnert, S.; Gillardon, F.; Hcngerer, B.; Otto, M., Neurochemical approaches in the laboratory diagnosis of Parkinson and Parkinson dementia syndromes: a review. CNS Neurosci Ther, 2009. 15, (2): p. 157-82; Parnetti, L., et al., Cerebrospinal fluid biomarkers in Parkinson's disease with dementia and dementia with Lewy bodies. Biol Psychiatry, 2008. 64(10): p. 850-5). However, for prognosis of disease progression in an individual patient this neurochemical profile is currently of limited use (Jesse, S. S., P.; Lehnert, S.; Gillardon, F.; Hengerer, B.; Otto, M., Neurochemical approaches in the laboratory diagnosis of Parkinson and Parkinson dementia syndromes: a review. CNS Neurosci Ther, 2009. 15, (2): p. 157-82).

Diagnosis and/or prognosis of PDD and differential diagnosis and/or prognosis between PDD and PD is still performed by physicians on the basis of the patient's medical history and neurological examination.

Thus, there is a need in the art for laboratory-based tests for the diagnosis and/or prognosis of PDD and for differential diagnosis and/or prognosis between PDD and PD.

Using an optimized protocol for the proteomic analysis of a cerebrospinal fluid sample (CSF), which particularly accounts for the brain protein variation caused by CSF flow (Brechlin, P., et al., Cerebrospinal fluid-optimized two-dimensional difference gel electrophoresis (2-D DIGE) facilitates the differential diagnosis of Creutzfeldt-Jakob disease. Proteomics, 2008. 8(20): p. 4357-66), the present inventors investigated a set of well defined clinical groups of patients with PD, PDD and a control group to find a biomarker which can differentiate between demented and non-demented persons.

Thereby, they found that PDD patients can be identified and PDD patients can be distinguished from PD patients by detecting O-glycosylation in a protein comprising a Ser/Thr motive, particularly Serpin A1, and/or by detecting the level of sialic acid on a protein comprising a Ser/Thr motive, particularly Serpin A1, in a biological sample such as a cerebrospinal fluid (CSF) sample.

### SUMMARY OF THE INVENTION

In a first aspect, the present invention relates to a method for diagnosing and/or prognosing Parkinson's disease dementia (PDD) or for differential diagnosing and/or prognosing between Parkinson's disease (PD) and Parkinson's disease dementia (PDD) comprising the steps of:
(i) detecting O-glycosylation in a protein comprising a Ser/Thr motive and/or the level of sialic acid on a protein comprising a Ser/Thr motive in a biological sample from a subject, and
(ii) identifying the subject as experiencing Parkinson's disease dementia (PDD) or being prone thereto, if O-glycosylation in said protein is present or increased and/or the level of sialic acid on said protein is increased in said biological sample compared to a control.

In a second aspect, the present invention relates to a molecule for detecting O-linked glycomoieties in a protein comprising a Ser/Thr motive and/or glycosylated isoforms of a Ser/Thr motive comprising protein for use in the diagnosis and/or prognosis of Parkinson's disease dementia (PDD) or differential diagnosis and/or prognosis between Parkinson's disease (PD) and Parkinson's disease dementia (PDD).

In a third aspect, the present invention relates to means for the diagnosis and/or prognosis of Parkinson's disease dementia (PDD) or for differential diagnosis and/or prognosis between Parkinson's disease (PD) and Parkinson's disease dementia (PDD) comprising at least one molecule according to the second aspect.

In a fourth aspect, the present invention relates to a kit for the diagnosis and/or prognosis of Parkinson's disease dementia (PDD) or for differential diagnosis and/or prognosis between Parkinson's disease (PD) and Parkinson's disease dementia (PDD) comprising
(i) a means for detecting O-glycosylation in a protein comprising a Ser/Thr motive and/or the level of sialic acid on said protein, and optionally
(ii) a data carrier, and/or
(iii) a container.

In a fifth aspect, the present invention relates to the use of the molecule according to the second aspect, the means according to the third aspect, or the kit according to the fourth aspect in the method according to the first aspect.

This summary of the invention does not necessarily describe all features of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

As used in this specification and the appended claims, the singular forms “a”, “an”, and “the” include plural referents, unless the content clearly dictates otherwise.

The inventors of the present invention surprisingly found that PDD patients can be identified and PDD patients can be distinguished from PD patients by detecting O-glycosylation in a protein comprising a Ser/Thr motive, particularly Serpin A1, and/or by detecting the level of sialic acid on a protein comprising a Ser/Thr motive, particularly Serpin A1, in a biological sample such as a cerebrospinal fluid (CSF) sample. Particularly, the inventors of the present invention surprisingly found that patients experiencing PDD can be identified on the basis of the level of sialic acid on Serpin 1A isoforms and/or on the basis of the number of Serpin A1 isoforms in a biological sample such as a cerebrospinal fluid (CSF) sample.

Thus, in a first aspect, the present invention relates to a method for diagnosing and/or prognosing Parkinson's disease dementia (PDD) or for differential diagnosing and/or prognosing between Parkinson's disease (PD) and Parkinson's disease dementia (PDD) comprising the steps of:
(i) detecting O-glycosylation in a protein comprising a Ser/Thr motive and/or the level of sialic acid on a protein comprising a Ser/Thr motive in a biological sample from a subject, and
   (ii) identifying the subject as experiencing Parkinson's disease dementia (PDD) or being prone thereto, if O-glycosylation in said protein is present or increased and/or the level of sialic acid on said protein is increased in said biological sample compared to a control.

The term “diagnosing PDD”, as used herein, means determining whether a subject shows signs of or suffers from PDD. The term “prognosing PDD”, as used herein, means predicting whether a subject will show signs of or suffer from PDD in the future, but preferably also means predicting the course of PDD of a subject already showing signs of or suffering from PDD. The terms “differential diagnosing” and “differential prognosing” between PD and PDD relate to the discrimination between both disease states based on observations made with respect to the O-glycosylation in a protein comprising a Scr/Thr motive, particularly Serpin A1, and/or the level of sialic acid on said protein, particularly Serpin A1.

The term “Parkinson's disease (PD)”, as used herein, refers to a chronic (persistent) disorder of part of the brain. It is named after the person who first described it. It mainly affects the way the brain co-ordinates the movements of the muscles in various parts of the body. The main symptoms of Parkinson's disease are, for example, stiffness, shaking (tremor), and slowness of movement. Symptoms typically become gradually worse over time.

The term “Parkinson's disease dementia (PDD)”, as used herein, denotes the impairment of one or more cognitive processes, particularly related to memory, in subjects showing signs of or suffering from Parkinson's disease, also known as Parkinson disease, Parkinson's, idiopathic parkinsonism, primary parkinsonism, PD or paralysis agitans.

The term “a protein comprising a Ser/Thr motive”, as used herein, refers to a protein comprising a protein-O-fucosyltransferase recognition site. Thus, said protein may be a substrate for a protein-O-fucosyltransferase. O-linked glycans/glycomoieties (also designated as O-linked polysaccharides or oligosaccharides) are usually attached to the polypeptide chain by a protein-O-fucosyltransferase through serine or threonine residues, O-linked glycosylation is a true post-translational event which occurs in the Golgi apparatus and which does not require a consensus sequence and no oligosaccharide precursor is required for protein transfer. The most common type of O-linked glycans contain an initial GalNAc residue (or Tn epitope), these are commonly referred to as mucin-type glycans. Other O-linked glycans include glucosamine, xylose, galactose, fucose, or manose as the initial sugar bound to the Ser/Thr residues. O-linked glycoproteins are usually large proteins (>200 kDa) that are commonly bianttennary with comparatively less branching than N-glycans (see, for example, Robert G. Spiro, “Protein glycosylation: nature, distribution, enzymatic formation, and disease implications of glycopeptides bonds”, Glycobiology, Vol. 12, No. 4, pp. 43R-56R, 2002).

The term “a protein comprising a Ser/Thr motive”, as used herein, may refer to a protein comprising a Ser motive, to a protein comprising a Thr motive or to a protein comprising a Ser and a Thr motive, i.e. a Ser and/or Thr motive.

The term “protein isoform”, as used herein, refers to any of several different forms of the same protein. Usually, different forms of a protein may be produced from related genes, or may arise from the same gene by alternative splicing. A large number of isoforms are caused by single-nucleotide polymorphisms (SNPs) or small genetic differences between alleles of the same gene. These occur at specific individual nucleotide positions within a gene. A protein isoform may further be characterized by its posttranslational modifications. The term “posttranslational modifications”, as used herein, refers to modifications of amino acids which may extend the range of functions of the protein by attaching it to other biochemical functional groups such as acetate, phosphate, various lipids and carbohydrates, by changing the chemical nature of an amino acid (e.g. citrullination) or by making structural changes, like the formation of disulfide bridges. Posttranslational modifications usually occur after translation. Well known posttranslational modifications are glycosylations or phosphorylations.

An isoform of a protein that differs only with respect to the number and/or type of attached glycan is herein referred to as “glycosylated isoform”. Glycoproteins often consist of a number of different glycoforms, with alterations in the attached saccharide or oligosaccharide. These modifications may result from differences in biosynthesis during the process of glycosylation, or due to the action of glycosidases or glycosyltransferases. Glycoforms may be detected through detailed chemical analysis of separated glycoforms, but more conveniently detected through differential reaction with lectins, as in lectin affinity chromatography and lectin affinity electrophoresis.

The term “Serpin”, as used herein, comprises Serpin family members characterized by common structural features. Serpins are usually comprised of three beta-sheets and eight or nine alpha-helices. Serpins also possess an exposed region termed the reactive centre loop (RCL) that, in inhibitory molecules, includes the specificity determining region and forms the initial interaction with the target protease (see, for example, Loebermann H, Tokuoka R, Deisenhofer J, Huber R. (1984). “Human alpha 1-proteinase inhibitor. Crystal structure analysis of two crystal modifications, molecular model and preliminary analysis of the implications for function”. J Mol Biol. 177 (3): 531-57. doi:10.1016/0022-2836(84)90298-5. PMID 6332197; Stein P E, Leslie A G, Finch J T, Turrell W G, McLaughlin P J, Carrell R W. (1990). “Crystal structure of ovalbumin as a model for the reactive centre of serpins”. Nature. 347 (6288): 99-102. doi:10.1038/347099a0. PMID 2395463). Preferably, the Serpin family comprises Serpin A1, Serpin A8 and Serpin F1 (see below).

The term “Serpin A1” (also designated as SERPINA1—serpin peptidase inhibitor, Glade A (alpha-1 antiproteinase, antitrypsin), member 1), as used herein, refers to a protease inhibitor belonging to the serpin (serine proteinase inhibitors) superfamily. It is also known as serum trypsin inhibitor. For most, the primary function of the Serpins is the regulation of proteolytic events associated with a plurality of biochemical pathways such as protein folding, cell migration, cell differentiation, modulation of inflammatory response, etc., but many have alternate functions such as hormone transport (cortisol-binding globulin and thyroxine binding globulin) or blood pressure regulation (angiotensinogen). Serpin A1 is secreted and its targets are, for example, elastase, plasmin, thrombin, trypsin, chymotrypsin, and plasminogen activator. Inhibitory Serpin A1 interacts with its target proteinase at a reactive site located within a loop structure in its C-terminal region. The reactive center loop (RCL) extends out from the body of the protein and directs binding to the target protease. The protease cleaves the Serpin A1 at the reactive site within the RCL, establishing a covalent linkage between the carboxyl group of the Serpin reactive site and the serine hydroxyl of the protease. The resulting inactive Serpin-protease complex is highly stable. The human gene encoding Serpin A1 is located at chromosome 14, region 14q32.1.

The term “Serpin A1 isoform”, as used herein, relates to any of several different forms of the same protein. From human (homo sapiens) Serpin A1, three isoforms produced by alternative splicing are known. Isoform 1 (SEQ ID NO: 1) has a length of 418 amino acids (canonical sequence) and a Mass of 46.73 kDa. Further, isoform 2 (SEQ ID NO: 2) has a length of 359 amino acids and a Mass of 40.26 kDa and differs from the amino acid sequence of isoform 1 in that amino acids 356 to 418 of isoform 1 are replaced by the amino acids VRSP (Val Arg Ser Pro) (SEQ ID NO: 4). Furthermore, isoform 3 (SEQ ID NO: 3) has a length of 306 amino acids and a Mass of 34.75 kDa and differs from the amino acid sequence of isoform 1 in that amino acids 307 to 418 of isoform 1 are missing.

The term “Serpin A1” encompasses Serpin A1 variants, e.g. all non-naturally or naturally occurring variants such as Serpin A1 homologues, particularly orthologues or paralogues. Preferably, the Serpin A1 variants have an amino acid sequence which is at least 60%, at least 70%, at least 75%, at least 80%, at least 85%, at least 90%, at least 95%, at least 99%, or 100% identical to SEQ ID NO: 1 (human Serpin A1 isoform 1), SEQ ID NO: 2 (human Serpin A1 isoform 2), or SEQ ID NO: 3 (human Serpin A1 isoform 3). For example, the Serpin A1 variants have an amino acid sequence which is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to SEQ ID NO: 1 (human Serpin A1 isoform 1), SEQ ID NO: 2 (human Serpin A1 isoform 2), or SEQ ID NO: 3 (human Serpin A1 isoform 3). Preferably, the sequence identity is over a continuous stretch of at least 20, 25, 30, 35, 40, 50, 60, 70, 80, 90, 100, 120, 150, 180, 200, 250, 300, 350, 400, or more amino acids, preferably over the whole length of the Serpin A1 amino acid sequence. As already mentioned above, alignment tools are well known to the person skilled in the art and can be, for example, obtained on the World Wide Web, e.g., ClustalW (www.ebi.ac.uk/clustalw) or Align (http://www.ebi.ac.uk/emboss/align/index.html) using standard settings, preferably for Align EMBOSS::needle, Matrix: Blosum62, Gap Open 10.0, Gap Extend 0.5.

Different proteins, particularly isoforms/glycosylated isoforms, can further be characterized by their posttranslational modifications such as glycosylations, e.g. N-linked glycosylations and/or O-linked glycosylations, in particular sialylations, i.e. protein modification with sialic acids.

The term “sialic acid”, as used herein, refers to an acidic sugar typically terminating the outer ends of glycan chains, such as N-glycan or O-glycan chains. Sialic acid is a generic term for the N- or O-substituted derivatives of neuraminic acid (NeuAc, 5-amino-3,5-dideoxy-D-glycero-D-galacto-non-2-ulosonic acid), a monosaccharide with a nine-carbon backbone. It is also the name for the most common member of this group, N-acetylneuraminic acid (Neu5Ac or NANA). The abbreviation for sialic acid(s) is “Sia(s)”.

The complete chemical names of Sias are too cumbersome for routine use. A uniform and simple nomenclature system is being increasingly used, in which the abbreviation Neu denotes the core structure neuraminic acid, and Kdn denotes the core structure 2-keto-3-deoxynononic acid. Various substitutions are then designated by letter codes (Ac=acetyl, Gc=glycolyl, Me=methyl, Lt=lactyl, S=sulfate), and these are listed along with numbers indicating their location relative to the carbon positions. For example, N-glycolylneuraminic acid is Neu5Gc or N-acetylneuraminic acid is Neu5Ac.

A terminal sialic acid (Sia), oligosialic acid (oligoSia), or polysialic acid (polySia) may exist on glycoproteins such as proteins comprising N-glycans or O-glycans. An oligosialic acid (oligoSia) is generally designated as an extended homopolymer of two sialic acid molecules, while a polysialic acid (polySia) is generally designated as an extended homopolymer of more than two sialic acid molecules found on glycoproteins (e.g. proteins comprising N-glycans or O-glycans). The linkages between the Sia units in an oligoSia chain or polySia chain can vary. The most common linkage is, however, a α2-8 linkage. Terminal sialic, oligosialic, and polysialic acids can, for example, be degraded by a sialidase (neuraminidase). Said enzyme is able to cleave α2-8 linkages between sialic acid residues or α2-3 linkages between a sialic acid residue and a hexose molecule such as a mannose, glucose or galactose molecule. The enzyme neuraminidase is able to remove terminal sialylation from N- and/or O-linked glycans.

For example, several isoforms of Serpin A1 achieved by posttranslational modifications are known, resulting from the combination of different N-linked glycan structures and mature N-terminus. For example, N-linked glycan at Asn107 is alternatively di-antennary, tri-antennary or tetra-antennary, whereas glycan at Asn70 is di-antennary with trace amounts of tri-antennary, and glycan at Asn271 is exclusively di-antennary. Thus, about 18 Serpin A1 isoforms carrying the above-mentioned posttranslational modifications may exist for Serpin A1 in humans. Said isoforms are preferably the followings:
(i) isoform 1 as mentioned above, more preferably in its mature form, with N-linked glycan at Asn107, which is di-antennary, whereas glycan at Asn70 is di-antennary, and glycan at Asn271 is exclusively di-antennary,
(ii) isoform 1 as mentioned above, more preferably in its mature form, with N-linked glycan at Asn107, which is tri-antennary, whereas glycan at Asn70 is di-antennary and glycan at Asn271 is exclusively di-antennary,
(iii) isoform 1 as mentioned above, more preferably in its mature form, with N-linked glycan at Asn107, which is tetra-antennary, whereas glycan at Asn70 is di-antennary and glycan at Asn271 is exclusively di-antennary,
(iv) isoform 1 as mentioned above, more preferably in its mature form, with N-linked glycan at Asn107, which is di-antennary, whereas glycan at Asn70 is di-antennary with trace amounts of tri-antennary, and glycan at Asn271 is exclusively di-antennary,
(v) isoform 1 as mentioned above, more preferably in its mature form, with N-linked glycan at Asn107, which is tri-antennary, whereas glycan at Asn70 is di-antennary with trace amounts of tri-antennary, and glycan at Asn271 is exclusively di-antennary,
(vi) isoform 1 as mentioned above, more preferably in its mature form, with N-linked glycan at Asn107, which is tetra-antennary, whereas glycan at Asn70 is di-antennary with trace amounts of tri-antennary, and glycan at Asn271 is exclusively di-antennary,
(vii) isoform 2 as mentioned above, more preferably in its mature form, with N-linked glycan at Asn107, which is di-antennary, whereas glycan at Asn70 is di-antennary, and glycan at Asn271 is exclusively di-antennary,
(viii) isoform 2 as mentioned above, more preferably in its mature form, with N-linked glycan at Asn107, which is tri-antennary, whereas glycan at Asn70 is di-antennary, and glycan at Asn271 is exclusively di-antennary,
(ix) isoform 2 as mentioned above, more preferably in its mature form, with N-linked glycan at Asn107, which is tetra-antennary, whereas glycan at Asn70 is di-antennary, and glycan at Asn271 is exclusively di-antennary,
(x) isoform 2 as mentioned above, more preferably in its mature form, with N-linked glycan at Asn107, which is di-antennary, whereas glycan at Asn70 is di-antennary with trace amounts of tri-antennary, and glycan at Asn271 is exclusively di-antennary,
(xi) isoform 2 as mentioned above, more preferably in its mature form, with N-linked glycan at Asn107, which is tri-antennary, whereas glycan at Asn70 is di-antennary with trace amounts of tri-antennary, and glycan at Asn271 is exclusively di-antennary,
(xii) isoform 2 as mentioned above, more preferably in its mature form, with N-linked glycan at Asn107, which is tetra-antennary, whereas glycan at Asn70 is di-antennary with trace amounts of tri-antennary, and glycan at Asn271 is exclusively di-antennary,
(xiii) isoform 3 as mentioned above, more preferably in its mature form, with N-linked glycan at Asn107, which is di-antennary, whereas glycan at Asn70 is di-antennary, and glycan at Asn271 is exclusively di-antennary,
(xiv) isoform 3 as mentioned above, more preferably in its mature form, with N-linked glycan at Asn107, which is tri-antennary, whereas glycan at Asn70 is di-antennary, and glycan at Asn271 is exclusively di-antennary,
(xv) isoform 3 as mentioned above, more preferably in its mature form, with N-linked glycan at Asn107, which is tetra-antennary, whereas glycan at Asn70 is di-antennary, and glycan at Asn271 is exclusively di-antennary,
(xvi) isoform 3 as mentioned above, more preferably in its mature form, with N-linked glycan at Asn107, which is di-antennary, whereas glycan at Asn70 is di-antennary with trace amounts of tri-antennary, and glycan at Asn271 is exclusively di-antennary,
(xvii) isoform 3 as mentioned above, more preferably in its mature form, with N-linked glycan at Asn107, which is tri-antennary, whereas glycan at Asn70 is di-antennary with trace amounts of tri-antennary, and glycan at Asn271 is exclusively di-antennary, and/or
(xviii) isoform 3 as mentioned above, more preferably in its mature form, with N-linked glycan at Asn107, which is tetra-antennary, whereas glycan at Asn70 is di-antennary with trace amounts of tri-antennary, and glycan at Asn271 is exclusively di-antennary.

Preferably, the mature form ranges from Glu25 to Lys418 of isoform 1 of Serpin A1 (SEQ ID NO: 1). Thus, the mature form of isoform 1 of Serpin A1 preferably lacks Met1 to Ala24 or amino acids corresponding thereto. The same applies to the mature forms of isoforms 2 and 3 of Serpin A1.

Proteolytic processing may yield the truncated form that ranges from Asp30 to Lys418 of isoform 1 of Serpin A1 Thus, the mature form of isoform 1 of Serpin A1 may lack Met1 to Gly29 or amino acids corresponding thereto. The same applies to the truncated forms of isoforms 2 and 3 of Serpin A1.

The structure of the antennas is preferably Neu5Ac(alpha1-6)Gal(beta1-4)GlcNAc attached to the core structure Man(alpha1-6)[Man(alpha1-3)]Man(beta1-4)GicNAc(beta1-4)GlcNAc. It should be noted that some antennas may be fucosylated, which forms a Lewis-X determinant.

The above-mentioned isoforms may be detected using a Serpin A1 specific antibody which binds to the N-terminal region of said Serpin A1 isoforms, preferably after performance of an isoelectric focusing (IEF) or 2D gel electrophoresis. It is preferred that said antibody binds to the N-terminal region between Glu25 to Leu200 or between Asp30 to Leu200, more preferably between Glu25 to Leu150 or between Asp30 to Leu150, even more preferred between Glu25 to Ile100 or between Asp30 to Ile100, and most preferably between Glu25 to Ser60 or between Asp30 to Ser60, of the amino acid sequence according to SEQ ID NO: 1 to 3, or at an amino acid position corresponding thereto. It is preferred that a monoclonal anti-human Serpin A1 antibody (Catalog Number MAB1268, RD System) is used for the detection of the above-mentioned isoforms.

In the context of the present invention, residues in two or more polypeptides are said to “correspond” to each other if the residues occupy an analogous position in the polypeptide structures. As is well known in the art, analogous positions in two or more polypeptides can be determined by aligning the polypeptide sequences based on amino acid sequence or structural similarities. Such alignment tools are well known to the person skilled in the art and can be, for example, obtained on the World Wide Web, e.g., ClustalW (www.ebi.ac.uk/clustalw) or Align (http://www.ebi.ac.uk/emboss/align/index.html) using standard settings, preferably for Align EMBOSS::needle, Matrix: Blosum62, Gap Open 10.0, Gap Extend 0.5. Those skilled in the art understand that it may be necessary to introduce gaps in either sequence to produce a satisfactory ali ent. Residues in two or more Serpin A1 isoforms are said to “correspond” if the residues are aligned in the best sequence alignment. The “best sequence alignment” between two polypeptides is defined as the alignment that produces the largest number of aligned identical residues. The “region of best sequence al ent” ends and, thus, determines the metes and bounds of the length of the comparison sequence for the purpose of the determination of the similarity score, if the sequence similarity, preferably identity, between two aligned sequences drops to less than 30%, preferably less than 20%, more preferably less than 10% over a length of 10, 20 or 30 amino acids.

As mentioned above, the protein which is detected in step (i) of the method of the present invention is an O-glycosylated protein comprising a Ser/Thr motive. In preferred embodiments of the method of the present invention, the O-glycosylated protein comprising a Ser/Thr motive comprises sialylated O-linked glycomoieties/glycans. In more preferred embodiments of the method of the present invention, said O-linked glycomoieties/glycans are hypersialylated. Particularly, the O-glycosylated protein comprising a Ser/Thr motive is Serpin A1, more particularly the O-glycosylated protein Serpin A1 comprises sialylated O-linked glycomoieties/glycans, and most particularly said O-linked glycomoieties/glycans are hypersialylated.

Detecting O-glycosylation in a protein comprising a Ser/Thr motive is carried out by art known methods. They include the analysis of glycomoieties with O-glycosylation specific antibodies, mass spectroscopy, e.g. MALDI-TOF analysis, and the determination of the number of glycosylated isoforms, preferably of Serpin A1 isoforms. Thus, in preferred embodiments of the method of the present invention, the detection of O-glycosylation comprises the step of determining the number of glycosylated isoforms. Particularly, the number of glycosylated isoforms of a protein comprising a Ser/Thr motive.

It should be noted that the detected number of glycosylated isoforms, which is indicative of the presence of Parkinson's disease dementia (PDD) or which allows differentiating between Parkinson's disease (PD) and Parkinson's disease dementia (PDD) may vary depending on the method used. However, independent from the method used for diagnosing and/prognosing PDD or for differential diagnosing and/or prognosing between PD and PDD, the number of glycosylated isoforms, preferably Serpin A1 isoforms, has to be increased by at least 1, preferably by at least 2, in the biological sample from a subject compared to a control in order that the subject is identified as experiencing PDD. Said additional glycosylated isoforms are preferably isoforms (e.g. Serpin A1 isoforms) comprising sialylated O-linked glycomoieties, particularly hypersialylated O-linked glycomoieties. Said additional glycosylated isoforms are preferably isoforms having an isoelectric point (pI) of 4.5 to 5, e.g. a pI of 4.5, 4.6, 4.7, 4.8, 4.9, or 5.0.

In particularly preferred embodiments of the method of the present invention, the determined glycosylated Serpin A isoform(s) abs (have) a molecule weight of between 38 and 45 kDa, e.g. 38, 39, 40, 41, 42, 43, 44, or 45 kDa, preferably 43 kDa.

Alternatively or additionally, the method of the present invention comprises the step of detecting the level of sialic acid on the Ser/Thr motive comprising protein, preferably on Serpin A1. The level of sialic acid on the respective Ser/Thr motive comprising protein, preferably on Serpin A1, which is indicative for the presence of Parkinson's disease dementia (PDD) or which allows differentiating between Parkinson's disease (PD) and Parkinson's disease dementia (PDD) may vary depending on the method used. However, independent from the method used for diagnosing and/prognosing PDD or for differential diagnosing and/or prognosing between PD and PDD, the level of sialic acid on the respective Ser/Thr motive comprising protein, preferably on Serpin A1, has to be increased by at least 1%, more preferably by at least 5% or 10% in the biological sample from a subject compared to a control in order that the subject is identified as experiencing PDD.

To assure the comparability of the results (values) determined by analyzing the biological sample from a subject to be tested, with the control results (values) determined by analyzing the biological sample from a control subject, both results (values) are preferably achieved with the same methods, more preferably carried out under the same method/process conditions. Thus, preferably, only results (values) achieved with the same methods and more preferably under comparable, most preferably identical method/process conditions are compared to identify a subject experiencing Parkinson's disease dementia (PDD).

The term “biological sample”, as used herein, refers to any biological sample comprising a protein comprising a Ser/Thr motive, e.g. Serpin A1, particularly Serpin A1 isoforms comprising sialic acid(s) on their glycan structure. The biological sample may be any sample comprising cells or the products of cells derived from a subject. It may be a body fluid sample, a tissue sample (e.g. explant or section), or a cell sample (e.g. cell(s) or cell colonies). For example, said biological sample may be an explant sample, a section sample, a single cell sample, a cell colony sample, a cell culture sample, a blood sample, an urine sample, or a sample from another peripheral source. The biological samples may be mixed or pooled, e.g. a biological sample may be a mixture of a blood sample and an urine sample. The biological sample may be provided by removing cell colonies, an explant, or a section from a subject, but may also be provided by using a previously isolated sample. For example, a tissue sample may be removed from a subject by conventional biopsy techniques or a blood sample may be taken from a subject by conventional blood collection techniques.

Preferably, the biological sample is a body fluid sample, a tissue sample, a cell colony sample, a single cell sample or a cell culture sample. More preferably, the tissue sample is a section or an explant sample. Said cells may be brain cells or said tissues may be derived from brain tissue, preferably from the substantia nigra. It is most particularly preferred that said cells are neurons and/or dopaminergic cells.

It is preferred that the tissue sample from a subject has a weight of between 0.1 and 500 mg, more preferably of between 0.5 and 250 mg, and most preferably of between 1 and 50 mg, i.e. 0.1, 0.2, 0.3, 0.4, 0.5, 0.6, 0.7, 0.8, 0.9, 1, 2, 2.5, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 mg.

It is further preferred that the cell sample (e.g. cell colony sample or cell culture sample) from a subject consists of between 10² and 10¹⁰ cells, more preferably of between 10³ and 10⁷ cells, and most preferably of between 10⁴ and 10⁶ cells, i.e. 10², 10³, 10⁴, 10⁵, 10⁶, 10⁷, 10⁸, 10⁹, or 10¹⁰ cells.

The term “body fluid sample”, as used herein, refers to a liquid sample derived from the body of a subject, e.g. human or animal. Said body fluid sample may be a blood, urine, sputum, breast milk, cerebrospinal fluid (CSF), cerumen (earwax), endolymph, perilymph, gastric juice, mucus, peritoneal fluid, pleural fluid, saliva, sebum (skin oil), or a sweat sample including components or fractions thereof. Preferably, it is a CSF sample, a blood sample, more preferably a whole blood sample or serum sample, an urine sample, or a saliva sample including components or fractions thereof, most preferably a CSF. A “body fluid sample” may be provided by removing a body liquid from a subject, but may also be provided by using previously isolated body fluid sample material. In the context of the present invention said “body fluid sample” may allow for a non-invasive diagnosis/and or prognosis of a subject. It is preferred that the body fluid sample from a subject has a volume of between 0.1 and 20 ml, more preferably of between 0.2 and 10 ml, more preferably between 0.4 and 8 ml and most preferably between 1 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

The “subject”, as mentioned in the method above, may be a subject which is suspected to experience PDD. The subject may be diagnosed to experience PDD. The “subject”, as mentioned in the method above, may also be a subject which already experiences PDD. Particularly, the subject may be retested for experiencing PDD and may be diagnosed to still experiencing PDD, e.g. a more severe or pronounced form, level or stage of PDD. The “subject”, as mentioned in the method above, may be a subject suspected to develop PDD. The subject may be prognosed to develop PDD in the future. Particularly, the subject may be a subject which already suffers from PD but which is suspected to have developed a dementia in the course of the disease. The “subject”, as mentioned in the method above, may further be a human or another mammal, e.g. a rodent (e.g. rat, hamster, or mouse) or monkey, or may be another animal than a mammal, e.g. an avian. In a preferred embodiment, the subject is a human or another mammal. The subject to be diagnosed and/or prognosed with the method of the present invention may also be designated as “test subject” herein.

The “control (value)”, as mentioned in the method above, may be a value/data of a (control) subject known to experience PD or PDD. The “control (value)”, as mentioned in the method above, may also refer to a value/data of a (control) subject known to not experience PD and/or PDD (negative control), i.e. being healthy. Particularly, the “control (value)”, as mentioned in the method above, may be (i) the number of O-linked glycomoieties comprised in said Ser/Thr motive comprising protein, preferably Serpin A1, and/or the number of glycosylated isoforms of said Ser/Thr motive comprising protein, preferably Serpin A1 isoforms, and/or the level of sialic acid on said Scr/Thr motive comprising protein, preferably Serpin A1, known to be present in a subject being healthy, i.e. not showing signs of or suffering from Parkinson's disease (PD) and/or Parkinson's disease dementia (PDD), (ii) the number of O-linked glycomoieties comprised in said Ser/Thr motive comprising protein, preferably Serpin A1, and/or the number of glycosylated isoforms of said Ser/Thr motive comprising protein, preferably Serpin A1 isoforms, and/or the level of sialic acid on said Ser/Thr motive comprising protein, preferably Serpin A1, known to be present in a subject showing signs of or suffering from Parkinson's disease dementia (PDD) (e.g. a specific form, level or stage of PDD), or (iii) the number of O-linked glycomoieties comprised in said Ser/Thr motive comprising protein, preferably Serpin A1, and/or the number of glycosylated isoforms of said Ser/Thr motive comprising protein, preferably Serpin A1 isoforms, and/or the level of sialic acid on said Ser/Thr motive comprising protein, preferably Serpin A1, known to be present in a subject showing signs of or suffering from Parkinson's disease (PD). In this respect, it should be noted that a subject having the above-mentioned disease or being healthy may also be designated as “control subject” herein. In this respect, it should be further noted that a “subject” that is known to be healthy, i.e. not suffering from PD and/or PDD, may possibly suffer from another disease not known/tested. Thus, a “healthy subject” has to be understood in the context of the present invention as a subject not suffering from PD and/or PDD, but possibly suffering from another disease not known/tested.

The inventors of the present invention found that with 2D gel electrophoreses, particularly with a 2D-immunoblot, it is possible to detect/determine the most abundant glycosylated isoforms, in particular Serpin A1 isoforms. For example, with the 2D gel electrophoreses, particularly 2D-immunoblot, the inventors of the present invention were able to determine (under the conditions elaborately described in the experimental part of the description) that the number of Serpin A1 isoforms in a healthy subject, i.e. in a subject not suffering from PDD and/or PD but possibly suffering from another disease not known/tested, or in a subject suffering from PD is ≦5 and that a test subject having >5 Serpin A1 isoforms, preferably 6 or 7 Serpin A1 isoforms, is identified as experiencing PDD. Preferably, increase by 1 or 2 Serpin A1 isoforms having (an) isoelectric point(s) (pI(s)) which is (are) lower than the isoelectric points (pIs) of the other Serpin A1 isoforms present in the control is detected. Said 1 or 2 additional Serpin A1 isoforms have preferably an isoelectric point (pI) of 4.5 to 5, e.g. a pI of 4.5, 4.6, 4.7, 4.8, 4.9, or 5.0

It is preferred that in a method for diagnosing and/or prognosing Parkinson's disease dementia (PDD) or for differential diagnosing and/or prognosing between Parkinson's disease (PD) and Parkinson's disease dementia (PDD), the control (value) is
(i) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a healthy subject,
(ii) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a subject experiencing Parkinson's disease dementia (PDD), and/or
(iii) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a subject experiencing Parkinson's disease (PD).

It is particularly preferred that in a method for differential diagnosing and/or prognosing between Parkinson's disease (PD) and Parkinson's disease dementia (PDD), the control (value) is
(i) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a subject experiencing Parkinson's disease (PD).

It is preferred that the O-linked glycomoieties comprised in said Ser/Thr motive comprising protein are sialylated O-linked glycomoieties. It is more preferred that said O-linked glycomoieties are hypersialylated. Particularly, the protein comprising a Ser/Thr motive is Serpin A1, more particularly the protein Seipin A1 comprises sialylated O-linked glycomoieties, and most particularly said O-linked glycomoieties are hypersialylated.

Preferably, the number of glycosylated isoforms, particularly Serpin A1 isoforms, and/or the level of sialic acid on the Ser/Thr motive comprising protein, particularly Serpin A1, of the above-mentioned control subjects and the number of glycosylated isoforms, particularly Serpin A1 isoforms, and/or the level of sialic acid on the Ser/Thr motive comprising protein, particularly Serpin A1, of the above-mentioned test subjects are determined in the same type of biological sample such as blood sample, for example, blood serum sample, blood plasma sample, CSF or blood cell (e.g. erythrocytes, leukocytes and/or thrombocytes) sample.

It is preferred that the above-mentioned control (value) is an average control (value), particularly an average control (value) of at least 2 to 40 (control) subjects, more preferably of at least 10 to 40 (control) subjects, and most preferably of at least 15 to 40 (control) subjects. It is also preferred that said (control) subjects are of the same species, have the same gender and/or a similar age or stage of life. The (control) subject may be a human or another mammal, e.g. a rodent (e.g. rat, hamster, or mouse) or monkey, or may be another animal than a mammal such as an avian. It is further preferred that the (control) subject is a human or another mammal.

In a preferred embodiment, the subject is identified as experiencing Parkinson's disease dementia (PDD), if the number of glycosylated isoforms, particularly Serpin A1 isoforms, is increased by at least 1, more preferably by at least 2, 3, 4, 5, 6, 7, 8, 9, 10, 11, 12, 13, 14, 15, 16, 17, 18, 19, 20 or more, and/or the level of sialic acid on Serpin A1 isoforms is increased by at least 1%, at least 5%, or at least 10%, more preferably at least 20%, at least 30%, at least 40% or at least 50%, still more preferably at least 60%, at least 70%, at least 80%, and most preferably at least 90%, 95%, 99% or 100% in the biological sample from the subject compared to a control. For example, the subject is identified as experiencing Parkinson's disease dementia (PDD), if the level of sialic acid on the Ser/Thr motive comprising protein, particularly on Serpin A1, is increased by at least 1, 2, 3, 4, 5, 6, 7, 8, 9, 10, 11, 12, 13, 14, 105, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% in the biological sample from the subject compared to a control.

In a particularly preferred embodiment, the subject is identified as experiencing Parkinson's disease dementia (PDD), if the number of Serpin A1 isoforms >5, preferably 6, 7 or 8, or is ≧6, preferably 6, 7 or 8. The number of Serpin A1 isoforms may also be >5.5, preferably 6, 6.5, 7, 7.5, or 8. This is preferably the case if the number of isoforms of Serpin A1 is determined using 2D gel electrophoresis, more preferably using 2D immunoblots, immobilized pH gradient (iPG) gels or iPG strips (see below).

In the context of the present invention, the term “increased compared to a control” may also mean increased compared to a level zero, e.g. a level of O-glycosylation of zero and/or a level of sialic acid of zero. This may be the case where a O-glycosylation and/or a specific sialic acid structure is not present in the biological sample from a control subject, e.g. subject known to be healthy or known to suffer from PD, but present in the biological sample from the test subject, e.g. subject which is suspected to experience PDD. Said glycosylation and/or structure may be detected with a specific antibody or lectin in the biological sample from the test subject, e.g. subject which is suspected to experience PDD, but may not be detected in the biological sample from the control subject, e.g. subject known to be healthy or known to suffer from PD. In this case, the test subject, e.g. subject which is suspected to experience PDD, is diagnosed and/or prognosed as experiencing PDD.

In a preferred embodiment of the method of the first aspect of the present invention, the O-glycosylation, particularly Serpin A1 O-glycosylation, the number of glycosylated isoforms, particularly Serpin A1 isoforms, and/or the level of sialic acid on the Ser/Thr motive comprising protein, particularly Serpin A 1, is determined using an immunoassay, gel electrophoresis, spectrometry or chromatography, or a combination thereof. Preferably, the immunoassay is an enzyme immunoassay, more preferably an ELISA, or a Western Blot (also designated as immunoblot). The gel electrophoresis preferably is 1D (One-dimensional) or 2D (Two-dimensional) gel electrophoresis. The spectrometry preferably is mass spectrometry (MS), more preferably tandem mass spectrometry (MS/MS). The chromatography preferably is liquid chromatography (LC, or alternative HPLC) or affinity chromatography, e.g. protein, particularly lectin, affinity chromatography. The chromatography is preferably combined with spectrometry, more preferably mass spectrometry (MS), and is even more preferably liquid chromatography-mass spectrometry (LC-MS, or alternative HPLC-MS) and most preferably liquid chromatography-tandem mass spectrometry (LC-MS/MS, or alternative HPLC-MS/MS). Liquid chromatography-mass spectrometry is an analytical chemistry technique that combines the physical separation capabilities of liquid chromatography (LC, or alternatively HPLC) with the mass analysis capabilities of mass spectrometry (MS). LC-MS is a powerful technique as it has very high sensitivity and selectivity. Generally its application is oriented towards the specific detection and potential identification of molecules in the presence of other molecules, e.g. in a complex mixture. The gel electrophoresis is preferably combined with an immunoassay and is more preferably a 2D immunoblot.

If 1D gel electrophoresis is used the biological sample is preferably beforehand purified, e.g. with affinity chromatography.

The term “1D (One-dimensional) gel electrophoresis”, as used herein, includes protein separation techniques such as Sodium dodecyl sulfate polyacrylamide gel electrophoresis (SDS-PAGE), native gel electrophoresis and isoelectric focusing. The SDS-PAGE, for example, is a technique for separating proteins based on their ability to move within an electrical current, which is a function of the length of their polypeptide chains or of their molecular weight. The addition of the SDS detergent to these samples gives the proteins the same electrical charge. SDS-PAGE allows for separation of proteins from a wide range of samples including cells, tissues and whole blood. Native Gel Electrophoresis, for example, is a technique used mainly in protein electrophoresis where the proteins are not denatured and therefore separated based on their charge-to-mass ratio. The main types of native gels used in protein electrophoresis are polyacrylamide gels and agarose gels. It should be noted that unlike SDS-PAGE type electrophoreses, native gel electrophoresis does not use a charged denaturing agent. The proteins being separated, therefore, differ in molecular mass and intrinsic charge and experience different electrophoretic forces dependent on the ratio of the two. Since the proteins remain in the native state they may be visualized not only by general protein staining reagents but also by specific enzyme-linked staining. Further, isoelectric focusing (IEF) (also known as electrofocusing), for example, is a technique to separate the proteins by isoelectric point. Thereby, a gradient of pH is applied to a gel and an electric potential is applied across the gel, making one end more positive than the other. At all pHs other than their isoelectric point, proteins will be charged. If the proteins are positively charged, they will be pulled towards the more negative end of the gel and if the proteins are negatively charged they will be pulled to the more positive end of the gel. The proteins applied in the IEF will move along the gel and will accumulate at their isoelectric point; that is, the point at which the overall charge on the protein is 0 (a neutral charge). IEF is preferably carried out using immobilized pH gradient (iPG) gels, or iPG strips, more preferably dry and rehydratable iPG strips. Microfluidic chip based isoelectric focusing may also be used (Sommer and Hatch, Electrophoresis. 2009 March; 30(5):742-5.). Preferably, the 1D gel electrophoresis is isoelectric focusing (IEF) or SDS-PAGE as first dimension to separate the proteins according to their isoelectric point (pI).

The term “2D (Two-dimensional) gel electrophoresis”, refers to a form of gel electrophoresis commonly used to analyze proteins in two dimensions. 2D gel electrophoresis, for example, begins with 1D electrophoresis but then separates the molecules by a second property in a direction 90 degrees from the first. In 1D electrophoresis, proteins are separated in one dimension, so that all the proteins/molecules will lie along a lane but that the molecules are spread out across a 2D gel. The two dimensions that proteins are separated into using this technique can be isoelectric point, protein complex mass in the native state, and protein mass. Preferably, the first dimension is isoelectric focusing (IEF) and the second dimension is SDS-PAGE. As to the separation of proteins by their isoelectric point (isoelectric focusing, IEF) and as to the separation of proteins by their mass (SDS-PAGE) it is referred to the explanations made above. Preferably, the 2D gel electrophoresis is isoelectric focusing (IEF) as first dimension and SDS-PAGE as second dimension to separate the proteins according to their isoelectric point (pI) and according to their protein mass.

The proteins separated with gel electrophoresis can then be detected by a variety of means known to the person skilled in the art. Preferably, silver and Coomassie Blue staining are used. In the case of silver staining, a silver colloid is applied to the gel. The silver binds to cysteine groups within the protein. The silver is darkened by exposure to ultra-violet light. The darkness of the silver can be related to the amount of silver and therefore the amount of protein at a given location on the gel. This measurement can only give approximate amounts, but is adequate for most purposes.

Western blotting, for example, is a technique which allows the detection of specific proteins (native or denatured) from extracts made from cells or tissues or body liquid samples, before or after any purification steps. Proteins are generally separated by size using gel electrophoresis before being transferred to a synthetic membrane (typically nitrocellulose or PVDF) via dry, semi-dry, or wet blotting methods. The membrane can then be probed using antibodies using methods similar to immunohistochemistry, but without a need for fixation. Detection is typically performed using reporter enzyme linked antibodies, e.g. peroxidase linked antibodies to catalyze a chemiluminescent reaction or alkaline phosphatase linked antibodies to catalyze a colorimetric reaction. Western blotting is a routine molecular biology method that can be used to semiquantitatively or quantitatively compare protein levels between extracts. The size separation prior to blotting allows the protein molecular weight to be gauged as compared with known molecular weight markers. Western blotting is an analytical technique used to detect specific proteins in a given sample of tissue homogenate or extract. It uses gel electrophoresis to separate proteins by the length of the polypeptide (denaturing conditions) or by the 3-D structure of the protein (native/non-denaturing conditions). The colorimetric detection method may depend on incubation of the Western blot with a substrate that reacts with the reporter enzyme (such as peroxidase) that is bound to the secondary antibody. This converts the soluble dye into an insoluble form of a different color that precipitates next to the enzyme and thereby stains the membrane. Development of the blot may be then stopped by washing away the soluble dye. Protein levels may be evaluated through densitometry (how intense the stain is) or spectrophotometry. Further, chemiluminescent detection methods may depend on incubation of the Western blot with a substrate that will luminesce when exposed to the reporter on the secondary antibody. The light is then detected by photographic film, and more recently by CCD cameras which capture a digital image of the Western blot. The image may be analysed by densitometry, which evaluates the relative amount of protein staining and quantifies the results in terms of optical density. Newer software allows further data analysis such as molecular weight analysis if appropriate standards are used.

The enzyme-linked immunosorbent assay or ELISA, for example, is a method for quantitatively or semi-quantitatively determining protein concentrations from blood plasma, serum or cell/tissue extracts in a multi-well plate format (usually 96-wells per plate). Broadly, proteins in solution are adsorbed to ELISA plates. Antibodies specific for the protein of interest are used to probe the plate. Background is minimized by optimizing blocking and washing methods (as for IHC), and specificity is ensured via the presence of positive and negative controls. Detection methods are usually colorimetric or chemiluminescence based.

The term “mass spectrometry (MS)”, as used herein, refers to the use of an ionization source to generate gas phase ions from a sample on a surface and detecting the gas phase ions with a mass spectrometer. The term “laser desorption mass spectrometry” refers to the use of a laser as an ionization source to generate gas phase ions from a sample on a surface and detecting the gas phase ions with a mass spectrometer. The mass spectrometry may be a matrix-assisted laser desorption/ionization mass spectrometry or MALDI. In MALDI, the analyte is typically mixed with a matrix material that, upon drying, co-crystallizes with the analyte. The matrix material absorbs energy from the energy source which otherwise would fragment the labile biomolecules or analytes. The mass spectrometry may also be a surface-enhanced laser desorption/ionization mass spectrometry or SELDI. In SELDI, the surface on which the analyte is applied plays an active role in the analyte capture and/or desorption. The biological sample used in the method of the first aspect of present invention may have undergone chromatographic or other chemical processing. In mass spectrometry the “apparent molecular mass” refers to the molecular mass (in Daltons)-to-charge value, m/z, of the detected ions. How the apparent molecular mass is derived is dependent upon the type of mass spectrometer used. With a time-of-flight mass spectrometer, the apparent molecular mass is a function of the time from ionization to detection. The term “signal” refers to any response generated by a biomolecule such as protein under investigation. For example, the term signal refers to the response generated by a biomolecule hitting the detector of a mass spectrometer. The signal intensity correlates with the amount or concentration of the biomolecule. The signal is defined by two values: an apparent molecular mass value and an intensity value generated as described. The mass value is an elemental characteristic of the biomolecule, whereas the intensity value accords to a certain amount or concentration of the biomolecule with the corresponding apparent molecular mass value. Thus, the “signal” always refers to the properties of the biomolecule.

The term “tandem mass spectrometry (MS/MS)”, as used herein, refers to multiple rounds of mass spectrometry, usually separated by some form of molecule fragmentation. For example, one mass analyzer can isolate one peptide from many entering a mass spectrometer. A second mass analyzer then stabilizes the peptide ions while they collide with a gas, causing them to fragment by collision-induced dissociation (CID). A third mass analyzer then sorts the fragments produced from the peptides. Tandem MS can also be done in a single mass analyzer over time, as in a quadrupole ion trap. There are various methods for fragmenting molecules for tandem MS, including collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD) and surface-induced dissociation (SID).

In a preferred embodiment, the mass spectrometry is an electrospray ionization mass spectrometry (ESI-MS), a matrix-assisted laser desorption/ionization mass spectrometry (MALDI-MS), or an electron capture dissociation mass spectrometry (ECD-MS).

In another preferred embodiment, the mass spectrometry employs tandem mass tags (TMT), isobaric tags for relative and absolute quantitation (iTRAQ), or isotope-coded affinity tags (ICATs).

In a preferred embodiment, the number of glycosylated isoforms, particularly Serpin A1 isoforms, is determined in a biological sample from a subject in step (i) of the method of the present invention
(a) using 2D gel electrophoresis with isoelectric focusing (IEF) as a first dimension and SDS-PAGE as a second dimension to separate the proteins according to their isoelectric point (PI) and their protein mass, and
(b) using silver or Coomassie Blue staining to allow detection of the separated proteins.

The number of glycosylated isoforms, particularly Serpin A1 isoforms, determined in step (i) is further compared in step (ii) to the number of glycosylated isoforms, particularly Serpin A1 isoforms, of a control, preferably obtained from a control subject using the same biological sample, the same detection method and under the same method conditions.

In a further preferred embodiment, the number of glycosylated isoforms, particularly Serpin A1 isoforms, is determined in a biological sample from a subject in step (i) of the method of the present invention
(a) using 2D gel electrophoresis with isoelectric focusing (IEF) as a first dimension and SDS-PAGE as a second dimension to separate the proteins according to their isoelectric point (PI) and their protein mass, and
(b) performing a Western Blot (immunoblot) with a primary antibody which binds to the amino acid sequence of any glycosylated isoforms, particularly Serpin A1 isoform, e.g. which binds to the sequence located within the N-region of any glycosylated isoforms, particularly Serpin A1 isoform, and with a secondary antibody which is labeled with a detectable label/tag, e.g. linked to a reporter enzyme such as alkaline phosphatase or horseradish peroxidase, and which binds to the primary antibody, and
(c) detecting the label/tag, for example, by carrying out chemiluminescent reactions (e.g. horseradish peroxidase) or colorimetric reactions (e.g. alkaline phosphatase).

The number of glycosylated iso o is, particularly Serpin A1 isoforms, determined in step (i) is er compared in step (ii) to the number of glycosylated isoforms, particularly Serpin A1 isoforms, of a control, preferably obtained from a control subject using the same biological sample, the same detection method and under the same method conditions.

In another preferred embodiment, the level of sialic acid on glycosylated isoforms, particularly Serpin A1 isoforms, is determined in a biological sample from a subject in step (i) of the method of the present invention
(a) using 1D gel electrophoresis with isoelectric focusing (IEF) as first dimension to separate the proteins according to their isoelectric point (PI), and
(b) performing a Western Blot (immunoblot) with a primary antibody which binds to sialic acid on glycosylated isoforms, particularly Serpin A1 isoforms, and with a secondary antibody which is labeled with alkaline phosphatase or horseradish peroxidase and which binds to the primary antibody,
(c) carrying out chemiluminescent reactions (e.g. horseradish peroxidase) or colorimetric reactions (e.g. alkaline phosphatase), and
(d) evaluating the level of sialic acid on glycosylated isoforms, particularly Serpin A1 isoforms, trough densitometry (how intense the stain/light is) or spectrophotometry.

The level of sialic acid on glycosylated isoforms, particularly Serpin A1 isoforms, determined in step (i) is further compared in step (ii) to the level of sialic acid on glycosylated isoforms, particularly Serpin A1 isoforms, of a control, preferably obtained from a control subject using the same biological sample, the same detection method and under the same method conditions.

In a further preferred embodiment, the level of sialic acid on glycosylated isoforms, particularly Serpin A1 isoforms, is determined in a biological sample from a subject in step (i) of the method of the present invention
(a) using 2D gel electrophoresis with isoelectric focusing (IEF) as a first dimension and SDS-PAGE as a second dimension to separate the proteins according to their isoelectric point (PI) and their protein mass,
(b) performing a Western Blot (immunoblot) with a primary antibody which binds to sialic acid on glycosylated isoforms, particularly Serpin A1 isoforms, and with a secondary antibody which is labeled with alkaline phosphatase or horseradish peroxidase and which binds to the primary antibody,
(c) carrying out chemiluminescent reactions (e.g. horseradish peroxidase) or colorimetric reactions (e g alkaline phosphatase) and
(d) evaluating the level of sialic acid on glycosylated isoforms, particularly Serpin A1 isoforms, trough densitometry (how intense the stain/light is) or spectrophotometry.

The level of sialic acid on glycosylated isoforms, particularly Serpin A1 isoforms, determined in step (i) is further compared in step (ii) to the level of sialic acid on glycosylated isoforms, particularly Serpin A1 isoforms, of a control, preferably obtained from a control subject using the same biological sample, the same detection method and under the same method conditions.

The above-mentioned antibodies may be antibodies according to the second aspect of the present invention (see below).

The above-mentioned 1D gel electrophoresis with isoelectric focusing (IEF) as first dimension to separate the glycosylated isoforms, particularly Serpin A1 isoforms, according to their isoelectric point (pI), or 2D gel electrophoresis with isoelectric focusing (IEF) as first dimension and SDS-PAGE as second dimension to separate the glycosylated isoforms, particularly Serpin A1 isoforms, according to their isoelectric point (pI) and protein mass may be further combined with a sialidase enzyme treatment.

The term “sialidase enzyme (also designated as neuraminidase enzyme)”, as used herein, refers to a glycoside hydrolase enzyme (EC 3.2.1.18) that cleaves the glycosidic linkages of neuraminic acids. Sialidase enzymes are a large family, found in a range of organisms. The most commonly known neuraminidase is the viral neuraminidase, a drug target for the prevention of influenza infection. Sialidases catalyze the hydrolysis of terminal sialic acid residues from the newly formed virions and from the host cell receptors. Sialidase activities include assistance in the mobility of virus particles through the respiratory tract mucus and in the elution of virion progeny from the infected cell.

Thus, before the biological sample is applied to 1D gel electrophoresis or 2D gel electrophoresis in step (i) of the method of the present invention, the biological sample may be splited in two parts. One part of the sample is beforehand treated with a sialidase enzyme and the other part not. Subsequently, 1D gel electrophoresis or 2D gel electrophoresis is carried out with both samples.

The enzyme sialidase (also designated as neuroamidase) is able to digest terminal sialic, oligosialic and polysialic acids and can, thus, be used for the additional detection of a shift of differentially sialylated Ser/Thr motive comprising proteins, particularly Serpin A1, towards a more basic isoelectric point (pI) on a 1D gel with IEF as first dimension or 2D gel with IEF as second dimension. In case of Serpin A1, the disappearance of hypersialylated Serpin A1 isoforms, preferably the Serpin A1 isoforms with the lowest pI (e.g. with an pI in the range of 4.5 and 5), more preferably the one or two Serpin A1 isoforms with the lowest pI (e.g. with an pI in the range of 4.5 and 5), compared to a control sample (not treated with sialidase enzyme), is a sign for the presence of PDD (see examples, particularly FIG. 5).

As mentioned above, the proteins applied in the IEF gel/strip will move dependent on their overall charge along the gel and will accumulate at their isoelectric point; that is, the point at which the overall charge on the protein is 0 (a neutral charge). If the enzyme sialidase is used, the following generally applies: Sialic acid leads to an acidification of the protein by giving it a more negative charge which in turn requires a more acidic pH to be neutralized in the IEF gel/strip. When the sialic acid residues are removed, the protein will be less negatively charged, thus, requiring a less acidic pH for neutralization. This leads to a shift to a more basic pI.

Accordingly, the above-described method preferably allows the determination of the number of Serpin A1 isoforms (in the presence of PDD >5 isoforms on an IEF gel or on a 2D gel with IEF as a second dimension) and/or the determination of a hypersialylated stage of Serpin A1 isoforms (pI shift to a more basic pI with disappearance of hypersialylated Serpin A1 isoforms, preferably the one or two Serpin A1 isoforms with the lowest pI).

It is preferred that the protein comprising a Ser/Thr motive is selected from the group consisting of a Serpin, preferably Serpin A1, Serpin A8, or Serpin F1; Fetuin A; Ceruloplasmin; Haptoglobin; and Zinc-alpha-2 glycoprotein. It is more preferred that the protein comprising a Ser/Thr motive is Serpin A1.

Preferably, Serpin A1 has an amino acid sequence according to SEQ ID NO: 1 (Isoform 1), SEQ ID NO: 2 (Isoform 2) or SEQ ID NO: 3 (Isoform 3) (all human, see above), Serpin A8 has an amino acid sequence according to SEQ ID NO: 24 (human), Fetuin A has an amino acid sequence according to SEQ ID NO: 25 (human), Ceruloplasmin has an Mo acid sequence according to SEQ ID NO: 26 (human), Serpin F1 has an amino acid sequence according to SEQ ID NO: 27 (human), Haptoglobin has an amino acid sequence according to SEQ ID NO: 28 (human), Zinc-alpha-2 glycoprotein has an amino acid sequence according to SEQ ID NO: 29 (human), or variants thereof.

The term “Serpin A8” encompasses Serpin A8 variants, e.g. all non-naturally or naturally occurring variants such as Serpin A8 homologues, particularly orthologues or paralogues. Preferably, the Serpin A8 variants have an amino acid sequence which is at least 60%, at least 70%, at least 75%, at least 80%, at least 85%, at least 90%, at least 95%, at least 99%, or 100% identical to SEQ ID NO: 24. For example, the Serpin A8 variants have an amino acid sequence which is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to SEQ ID NO: 24. Preferably, the sequence identity is over a continuous stretch of at least 20, 25, 30, 35, 40, 50, 60, 70, 80, 90, 100, 120, 150, 180, 200, 250, 300, 350, 400, or more no acids, preferably over the whole length of the Serpin A8 amino acid sequence.

The term “Fetuin A” encompasses FetuinA variants, e.g. all non-naturally or naturally occurring variants such as Fetuin A homologues, particularly orthologues or paralogues. Preferably, the Fetuin A variants have an amino acid sequence which is at least 60%, at least 70%, at least 75%, at least 80%, at least 85%, at least 90%, at least 95%, at least 99%, or 100% identical to SEQ ID NO: 25. For example, the Fetuin A variants have an amino acid sequence which is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to SEQ ID NO: 25. Preferably, the sequence identity is over a continuous stretch of at least 20, 25, 30, 35, 40, 50, 60, 70, 80, 90, 100, 120, 150, 180, 200, 250, 300, 350, or more amino acids, preferably over the whole length of the Fetuin A amino acid sequence.

The term “Ceruloplasmin” encompasses Ceruloplasmin variants, e.g. all non-naturally or naturally occurring variants such as Ceruloplasmin homologues, particularly orthologues or paralogues. Preferably, the Ceruloplasmin variants have an amino acid sequence which is at least 60%, at least 70%, at least 75%, at least 80%, at least 85%, at least 90%, at least 95%, at least 99%, or 100% identical to SEQ ID NO: 26. For example, the Ceruloplasmin variants have an amino acid sequence which is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to SEQ ID NO: 26. Preferably, the sequence identity is over a continuous stretch of at least 20, 25, 30, 35, 40, 50, 60, 70, 80, 90, 100, 120, 150, 180, 200, 250, 300, 350, 400, 500, 600, 700, 800, 900, 1000, or more amino acids, preferably over the whole length of the Ceruloplasmin amino acid sequence.

The term “Serpin F1” encompasses Serpin F1 variants, e.g. all non-naturally or naturally occurring variants such as Serpin F1 homologues, particularly orthologues or paralogues. Preferably, the Serpin F1 variants have an amino acid sequence which is at least 60%, at least 70%, at least 75%, at least 80%, at least 85%, at least 90%, at least 95%, at least 99%, or 100% identical to SEQ ID NO: 27. For example, the Serpin F1 variants have an amino acid sequence which is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to SEQ ID NO: 27. Preferably, the sequence identity is over a continuous stretch of at least 20, 25, 30, 35, 40, 50, 60, 70, 80, 90, 100, 120, 150, 180, 200, 250, 300, 350, 400, or more amino acids, preferably over the whole length of the Serpin F1 amino acid sequence.

The term “Haptoglobin” encompasses Haptoglobin variants, e.g. all non-naturally or naturally occurring variants such as Haptoglobin homologues, particularly orthologues or paralogues. Preferably, the Haptoglobin variants have an amino acid sequence which is at least 60%, at least 70%, at least 75%, at least 80%, at least 85%, at least 90%, at least 95%, at least 99%, or 100% identical to SEQ ID NO: 28. For example, the Haptoglobin variants have an amino acid sequence which is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to SEQ ID NO: 28. Preferably, the sequence identity is over a continuous stretch of at least 20, 25, 30, 35, 40, 50, 60, 70, 80, 90, 100, 120, 150, 180, 200, 250, 300, 350, 400, or more amino acids, preferably over the whole length of the Haptoglobin amino acid sequence.

The term “Zinc-alpha-2 glycoprotein” encompasses Zinc-alpha-2 glycoprotein variants, e.g. all non-naturally or naturally occurring variants such as Zinc-alpha-2 glycoprotein homologues, particularly orthologues or paralogues. Preferably, the Zinc-alpha-2 glycoprotein variants have an amino acid sequence which is at least 60%, at least 70%, at least 75%, at least 80%, at least 85%, at least 90%, at least 95%, at least 99%, or 100% identical to SEQ ID NO: 29. For example, the Zinc-alpha-2 glycoprotein variants have an amino acid sequence which is at least 60, 61, 62, 63, 64, 65, 66, 67, 68, 69, 70, 71, 72, 73, 74, 75, 76, 77, 78, 79, 80, 81, 83, 84, 85, 86, 67, 88, 89, 90, 91, 92, 93, 94, 95, 96, 97, 98, 99 or 100% identical to SEQ ID NO: 29. Preferably, the sequence identity is over a continuous stretch of at least 20, 25, 30, 35, 40, 50, 60, 70, 80, 90, 100, 120, 150, 180, 200, 220, 250, 270, or more amino acids, preferably over the whole length of the Zinc-alpha-2 glycoprotein amino acid sequence.

As already mentioned above, alignment tools are well known to the person skilled in the art and can be, for example, obtained on the World Wide Web, e.g., ClustalW (www.ebi.ac.uk/clustalw) or Align (http://www.ebi.ac.uk/emboss/align/index.html) using standard settings, preferably for Align EMBOSS::needle, Matrix: Blosum62, Gap Open 10.0, Gap Extend 0.5.

Preferably, the method of the first aspect of the present invention has a sensitivity, preferably a diagnostic sensitivity, of at least 70%, at least 75%, at least 80%, at least 85%, at least 90%, at least 95%, or at least 99% for PDD, and more preferably of 100% for PDD, e.g. of at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 or of 100% for PDD.

The term “sensitivity”, as used herein, means a statistical measure of how well a classification test correctly identifies a condition, for example how frequently it correctly classifies PDD. An optimal prediction can achieve 100% sensitivity (i.e. predict all patients from the PDD group as suffering from PDD).

Further, it is preferred that the diagnosis comprises (i) determining the presence or occurrence of PDD, (ii) monitoring the course of PDD, (iii) staging of PDD, (iv) measuring the response of a subject with PDD to therapeutic intervention, and/or (v) classification of a subject with PDD. It is further preferred that the prognosis comprises (i) predicting or estimating the occurrence, preferably the severity of occurrence, of PDD, and/or (ii) predicting or estimating the response of a subject with PDD to therapeutic intervention.

In a preferred embodiment of the method of the present invention, the O-glycosylation in one or more, e.g. 1, 2, 3, 4, 5, 6, 7, 8, 9, or 10, proteins comprising a Ser/Thr motive and/or the level of sialic acid on one or more, e.g. 1, 2, 3, 4, 5, 6, 7, 8, 9, or 10, proteins comprising a Ser/Thr motive is detected. Said one or more proteins are preferably selected from the group consisting of a Serpin, preferably Serpin A1, Serpin A8, or Serpin F1; Fetuin A; Ceruloplasmin; Haptoglohin; and Zinc-alpha-2 glycoprotein (see above).

In a second aspect, the present invention relates to a molecule for detecting O-linked glycomoieties in a protein comprising a Ser/Thr motive and/or a glycosylated isoform(s) of a Ser/Thr motive comprising protein for (use in) the diagnosis and/or prognosis of Parkinson's disease dementia (PDD) or differential diagnosis and/or prognosis between Parkinson's disease (PD) and Parkinson's disease dementia (PDD).

With respect to the meaning and preferred embodiments of the terms “biological sample”, “protein comprising a Ser/Thr motive”, and “subject” it is referred to the definitions and explanations provided above. Also all other terms used in the description of this aspect have the meaning as described above.

Said molecule may detect the amino acid sequence of a protein comprising a Ser/Thr motive, particularly Serpin A1, (e.g. the epitope(s), also known as antigenic determinant(s)) and/or the glycan structure/glycomoieties on a protein comprising a Ser/Thr motive, particularly Serpin A1. The detected glycan structure/glycomoieties may comprise sialic acid or may not comprise sialic acid. Said molecule may also detect the amino acid sequence of a protein comprising a Ser/Thr motive, particularly Serpin A1, (e.g. the epitope(s), also known as antigenic determinant(s)) and/or sialic acid on a protein comprising a Ser/Thr motive, particularly Serpin A1. A molecule detecting sialic acid on a protein comprising a Ser/Thr motive, particularly the Serpin A1, may exclusively detect sialic acid(s) or may also detect glycan structures/glycomoieties adjacent to the sialic acid(s). Further, a molecule detecting sialic acid may detect one or more sialic acid(s), e.g. (a) terminal sialic acid(s), oligosialic acids and/or polysialic acids. Preferably, said molecule detects the amino acid sequence of a protein comprising a Ser/Thr motive, particularly Serpin A1 (e.g. the epitope(s), also known as antigenic determinant(s)) and/or sialic acid on a protein comprising a Ser/Thr motive, particularly Serpin A1.

It is preferred that the above-mentioned molecule allows the detection/determination of O-glycosylation in a protein comprising a Ser/Thr motive, particularly Serpin A1, the number of glycosylated isoforms, particularly Serpin A1 isoforms, and/or the level of sialic acid on a protein comprising a Ser/Thr motive, particularly Serpin A1, preferably in a biological sample from a subject and, thus, the diagnosis and/or prognosis of PDD or the differential diagnosis and/or prognosis between PD and PDD. It is particularly preferred that said molecule is able to detect/determine all glycosylated isoforms of a protein comprising a Ser/Thr motive, particularly all Serpin A1 isoforms, which may be present in the biological sample and/or is able to detect/determine all sialic acid(s) (the sialic acid structure) which may be present on a protein comprising a Ser/Thr motive, particularly Serpin A1. Said molecule may also be able to detect/determine only a specific glycosylated isoform, particularly Serpin A1 isoform, which may be present in the biological sample and/or is able to detect/determine only a specific sialic acid structure which may be present on a protein comprising a Ser/Thr motive, particularly Serpin A1.

It is preferred that a molecule which allows the detection/determination of the level of sialic acid on a protein comprising a Ser/Thr motive, particularly Serpin A1, is a molecule which exclusively detects sialic acid(s) or which detects sialic acid(s) and glycan structures/glycomoieties adjacent to the sialic acid(s).

It is also preferred that a molecule which allows the detection/determination of the number of glycosylated isoforms of a protein comprising a Ser/Thr motive, particularly Serpin A1 isoforms, is a molecule which detects the amino acid sequence of a protein comprising a Ser/Thr motive, particularly Serpin A1, (e.g. the epitope(s), also known as antigenic determinant(s)).

The above-mentioned molecule may be a protein such as an antibody or a lectin, a polypeptide such as an antibody fragment, a peptide such as a mass spectrometry probe, or a small molecule.

The term “peptide”, as used herein, refers to a short polymer of amino acids linked by peptide bonds. It has the same peptide bonds as those in proteins, but is commonly shorter in length. The shortest peptide is a dipeptide, consisting of two amino acids joined by a single peptide bond. There can also be a tripeptide, tetrapeptide, pentapeptide, etc. A peptide has an amino end and a carboxyl end, unless it is a cyclic peptide. Preferably, a peptide has a length of between 2 to 20 amino acids, more preferably of between 5 to 20 amino acids and most preferably of between 7 to 15 amino acids, e.g. 2, 3, 4, 5, 6, 7, 8, 9, 10, 11, 12, 13, 14, 15, 16, 17, 18, 19, or 20 amino acids.

The term “polypeptide”, as used herein, refers to a part of a protein which is composed of a single linear chain of amino acids bonded together by peptide bonds. Preferably, the polypeptide has a length of more than 20 amino acids, more than 30 amino acids, or more than 40 amino acids. More preferably, the polypeptide has a length of between 21 and 200 amino acids, most preferably of between 50 and 100 amino acids, e.g. 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, or 200 amino acids.

The term “protein”, as used herein, may refer to a protein which comprises one or more polypeptides that resume a secondary and tertiary structure and additionally refers to a protein that is made up of several amino acid chains, i.e. several subunits, forming quaternary structures. The protein has sometimes non-peptide groups attached, which can be called prosthetic groups or cofactors.

The term “small molecule”, as used herein, refers to a low molecular weight organic compound which is by definition not a polymer. A small molecule may bind with high affinity to a biopolymer such as a protein and, thus, may allow the detection of said biopolymer. The upper molecular weight limit for a small molecule is usually about 800 Daltons.

Preferably, the molecule detects the amino acid sequence of a protein comprising a Ser/Thr motive, particularly Serpin A 1, (e.g. the epitope(s), also known as antigenic determinant(s)) and/or the glycan structure/glycomoieties on a protein comprising a Ser/Thr motive, particularly Serpin A1, by binding to said protein, particularly Serpin A1. Said molecule may bind to the amino acid sequence of a protein comprising a Ser/Thr motive, particularly Serpin A1, (e.g. the epitope(s), also known as antigenic determinant(s)) and/or the glycan structure/glycomoieties on a protein comprising a Ser/Thr motive, particularly Serpin A1. The bond glycan structure/glycomoieties may comprise sialic acid or may not comprise sialic acid. Said molecule may also bind to the amino acid sequence of a protein comprising a Ser/Thr motive, particularly Serpin A1 (e.g. the epitope(s), also known as antigenic determinant(s)) and/or sialic acid on a protein comprising a Ser/Thr motive, particularly Serpin A1. A molecule binding sialic acid on a protein comprising a Ser/Thr motive, particularly the Serpin A1, may exclusively bind sialic acid(s) or may also bind glycan structures/glycomoieties adjacent to the sialic acid(s). Further, a molecule binding sialic acid may bind one or more sialic acid(s), e.g. (a) terminal sialic acid(s), oligosialic acids and/or polysialic acids. More preferably, said molecule binds to the amino acid sequence of a protein comprising a Ser/Thr motive, particularly Serpin A1 (e.g. the epitope(s), also known as antigenic determinant(s)) and/or to the sialic acid on a protein comprising a Ser/Thr motive, particularly Serpin A1. Thus, for example, said molecule is a molecule that binds to the amino acid sequence of a protein comprising a Ser/Thr motive, particularly Serpin A1, that binds to the sialic acid(s) on a protein comprising a Ser/Thr motive, particularly Serpin A1, or that binds to both at the same time.

It is preferred that the above-mentioned molecule allows the detection/determination of O-glycosylation in a protein comprising a Ser/Thr motive, particularly Serpin A1, the number of glycosylated isoforms, particularly Serpin A1 isoforms, and/or the level of sialic acid on a protein comprising a Ser/Thr motive, particularly Serpin A1 isoforms, preferably in a biological sample from a subject and, thus, the diagnosis and/or prognosis of PDD or the differential diagnosis and/or prognosis between PD and PDD. It is particularly preferred that said molecule is able to bind all glycosylated isoforms of a protein comprising a Ser/Thr motive, particularly all Serpin A1 isoforms, which may be present in the biological sample and/or is able to bind all sialic acid(s) (the sialic acid structure) which may be present on a protein comprising a Ser/Thr motive, particularly Serpin A1. Said molecule may also be able to bind only a specific glycosylated isoform, particularly Serpin A1 isoform, which may be present in the biological sample and/or is able to bind only a specific sialic acid structure which may be present on a protein comprising a Ser/Thr motive, particularly Serpin A1.

It is preferred that a molecule which allows the detection/determination of the level of sialic acid on a protein comprising a Ser/Thr motive, particularly Serpin A1, is a molecule which exclusively binds to sialic acid(s) or which binds to sialic acid(s) and glycan structures/glycomoieties adjacent to the sialic acid(s).

It is also preferred that a molecule which allows the detection/determination of the number of glycosylated isoforms of a protein comprising a Ser/Thr motive, particularly Serpin A1 isoforms, is a molecule which binds to the amino acid sequence of a protein comprising a Ser/Thr motive, particularly Serpin A1, (e.g. the epitope(s), also known as antigenic determinant(s)).

The above-mentioned molecule may be a protein such as an antibody or a lectin, a polypeptide such as an antibody fragment, a peptide such as a mass spectrometry probe, or a small molecule.

It is particularly preferred that
(i) the sialic acid binding molecule is a sialic acid specific antibody or a fragment thereof (e.g. the variable region comprising the antigen binding site), a synthetic polypeptide, a recombinant polypeptide, preferably a darpin or an anticalin, a lectin, or a small molecule, or
(ii) the amino acid sequence binding molecule is a protein comprising a Ser/Thr motive (e.g. Serpin A1) specific antibody or a fragment thereof (e.g. the variable region comprising the antigen binding site), a synthetic polypeptide, a recombinant polypeptide, preferably a darpin or an anticalin, a lectin, or a small molecule.

It is more particularly preferred that the sialic specific antibody or fragment thereof, the synthetic polypeptide, recombinant polypeptide, preferably the darpin or anticalin, the lectin, or the small molecule binds to the sialic acid(s) as well as to glycan structures adjacent to the sialic acid(s).

The term “synthetic peptide or polypeptide”, as used herein, refers to a synthetically produced peptide or polypeptide. Generally, a peptide or polypeptide is synthetically produced by adding the amino acid from the carboxylate groups forward, as opposed to ribosomal production, wherein synthesizing starts with the amino group. For example, a synthetic polypeptide or peptide may be produced using liquid-phase synthesis or solid-phase peptide synthesis (SPPS), preferably Fmoc or Boc.

The term “recombinant peptide or polypeptide”, as used herein, refers to a genetically engineered polypeptide or peptide, i.e. a polypeptide or peptide with a sequence manipulated by man. Usually a recombinant polypeptide or peptide is produced from recombinant DNA (e.g. DNA coding for said polypeptide or peptide comprised in a vector such as an expression vector), for example, in a host organism such as a bacterial cell or yeast cell.

The term “darpin”, as used herein, refers to a genetically engineered antibody mimetic protein typically exhibiting highly specific and high-affinity target protein binding. It is derived from natural yrin proteins and consists of at least three, usually four or five repeat motifs of these proteins. Its molecular mass is about 14 or 18 kDa for four- or five-repeat DARPins, respectively.

The term “anticalin”, as used herein, refers to an artificial protein that is able to bind to antigens, e.g. to proteins or to small molecules, and is a type of antibody mimetic. It is derived from human lipocalins which are a family of naturally binding proteins. The size is about 180 amino acids and the mass is about 20 kDa.

The term “lectin”, as used herein, refers to a sugar-binding protein that is highly specific for its sugar moieties.

The synthetic polypeptides or peptides, recombinant polypeptides or peptides, preferably darpins or anticalins, lectins or small molecules according to the invention may be selected by routine screening of existing libraries, e.g. small molecule libraries. Suitable standard screening methods, e.g. phage display for polypeptides or peptides, are well known to the person skilled in the art. That said molecules are able to detect/determine glycosylated isoforms, particularly Serpin A1 isoforms, e.g. by binding to said isoforms, particularly Serpin A1 isoforms, can easily be tested by the person skilled in the art with methods known to the person skilled in the art, e.g. by fluorescence resonance energy transfer (FRET), co-immunoprecipitation or an Enzyme-linked immunosorbent assay (ELISA), also known as an enzyme immunoassay (EIA).

In one embodiment, the binding of the above-mentioned molecules to the Serpin A1 isoforms may be analyzed in form of an enzyme-linked immunosorbent assay (ELISA)-based experiment. Therefore, the Serpin A1 isoforms may be immobilized on the surface of an ELISA plate and contacted with the above-mentioned molecules. Binding of the molecules may be verified, for example, for proteins, polypeptides, peptides, and epitope-tagged compounds, by antibodies specific for said molecules or the epitope-tag. These antibodies might be directly coupled to an enzyme or detected with a secondary antibody coupled to said enzyme that—in combination with the appropriate substrates—carries out chemiluminescent reactions (e.g. horseradish peroxidase) or colorimetric reactions (e.g. alkaline phosphatase). In another embodiment, binding of molecules that cannot be detected by antibodies might be verified by labels directly coupled to the molecules. Such labels may include enzymatic labels, radioisotope or radioactive compounds or elements, fluorescent compounds or metals, chemiluminescent compounds and bioluminescent compounds. In another embodiment, the above-mentioned molecules might be immobilized on the ELISA plate and contacted with the Serpin A1 isoforms. Binding of said isoform may be verified by an antibody specific for said isoforms and chemiluminescence or colorimetric reactions as described above.

The term “antibody or fragment thereof”, as used herein, refers to immunoglobulin molecules and immunologically active portions of immunoglobulin molecules, i.e. molecules that contain an antigen binding site that specifically binds an antigen. Also comprised are immunoglobulin-like proteins that are selected through techniques including, for example, phage display to specifically bind to a target molecule or target protein. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. The “antibodies and fragments thereof” include, but are not limited to, polyclonal, monoclonal, monovalent, bispecific, heteroconjugate, multispecific, human, humanized (in particular CDR-grafted), deimmunized, or chimeric antibodies, single chain antibodies (e.g. scFv), Fab fragments, F(ab′)₂ fragments, fragments produced by a Fab expression library, diabodies or tetrabodies (Holliger P. et al., 1993), nanobodies, anti-idiotypic (anti-Id) antibodies, and epitope-binding fragments of any of the above.

In some embodiments, the antibody fragments are mammalian, preferably human antigen-binding antibody fragments and include, but are not limited to, Fab, Fab′ and F(ab′)₂, Fd, single-chain Fvs (scFv), single-chain antibodies, disulfide-linked Fvs (dsFv) and fragments comprising either a VL or VH domain. Antigen-binding antibody fragments, including single-chain antibodies, may comprise the variable domain(s) alone or in combination with the entirety or a portion of the following: hinge region, CL, CH1, CH2, and CH3 domains. The antigen-binding fragments may also comprise any combination of variable domain(s) with a hinge region, CL, CH1, CH2, and CH3 domains.

Antibodies usable in the invention may be from any animal origin including birds and mammals. Preferably, the antibodies are human, simian (e.g. chimpanzee, bonobo, macaque), rodent (e.g. mouse and rat), donkey, sheep rabbit, goat, guinea pig, camel, horse, or chicken. It is particularly preferred that the antibodies are of human or murine origin. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins.

Antibodies according to the second aspect of the invention may be produced by methods well known in the art or may simply be ordered to be made commercially. For example, an antibody according to the second aspect of the invention may be produced using the method described in US 2011/0034676 A1, wherein antibodies raised against sialylated Serpin A1 may be screened for binding to a particular Serpin A1 isoform, preferably to an antigen related to a Serpin A1/sialic acid conjugation specific to a particular Serpin A1 isoform. Means of preparing and characterizing antibodies or antibody fragments are also well known in the art (see, for example, Antibodies: A Laboratory Manual, Cold Spring Harbor Laboratory, 1988; incorporated herein by reference).

The antibody that recognizes the target antigen/protein, e.g. the Serpin A1 isoform, particularly a sialylated Serpin A1 isoform, is generally called the “primary antibody”. Said antibody may be labeled with a detectable tag/label in order to allow direct detection of the target antigen. Said detectable tag/label may be an enzymatic, fluorescent or radioisotope tag/label. Usually, however, the primary antibody is not labeled for direct detection. Instead a “secondary antibody” that has been labeled with a detectable tag/label (e.g. enzymatic, fluorescent or radioisotope tag/label) is applied in a second step to probe for the primary antibody, which is bound to the target antigen. For example, the primary antibody or the secondary antibody may be labeled with an affinity tag such as biotin.

It is preferred that the Serpin A1 specific molecule, preferably Serpin A1 specific antibody detects, particularly binds to, the N-terminal region of the Serpin A1 isoform, preferably to the N-terminal region of any Serpin A1 isoform, e.g. any isoform which may be present in the biological sample from a subject. It is more preferred that said molecule, preferably antibody, binds to the N-terminal region between Glu25 to Leu200 or between Asp30 to Leu200, more preferably between Glu25 to Leu150 or between Asp30 to Leu150, even more preferred between Glu25 to Ile100 or between Asp30 to Ile100, and most preferably between Glu25 to Ser60 or between Asp30 to Ser60, of the amino acid sequence according to SEQ ID NO: 1 to 3, or at an amino acid position corresponding thereto. It is particularly preferred that the antibody is a monoclonal anti-human Serpin A1 antibody (Catalog Number B1268, RD System).

It is er preferred that the molecule, preferably antibody, specific for sialic acid on Serpin A1 isoforms detects, particularly binds, the Neu5Ac(alpha1-6)Gal(beta1-4)GlcNAc structure encompassing sialic acid residues. It is more preferred that said molecule, preferably antibody, detects, particularly binds to, Neu5Ac(alpha1-6)Gal(beta1-4)GlcNAc and to the core structure Man(alpha1-6)[Man(alpha1-3)]Man(beta1-4)GlcNAc(beta1-4)GlcNAc of Serpin A1.

The Serpin A1 specific molecule, e.g. antibody, may also be a molecule, e.g. an antibody, which detects, particularly binds to, the amino acid sequence of only a specific Serpin A1 isoform, e.g. the N-terminal and/or C-terminal region of only a specific Serpin A1 isoform, e.g. only a specific Serpin A1 isoform which may be present in the biological sample from a subject.

For detection purposes, the molecule of the second aspect may be directly or indirectly labeled, e.g. with biotin to which a streptavidin complex may bind. The term “label”, as used herein, means a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and other entities which are or can be made detectable.

It is also preferred that the polypeptide or peptide for detecting a protein comprising a Ser/Thr motive, particularly Serpin A1, preferably the amino acid sequence of a protein comprising a Ser/Thr motive, particularly Serpin A1, is a mass spectrometry probe (peptide). The terms mass spectrometry probe or mass spectrometry peptide are interchangeable used herein. Said probe is a synthetic peptide or polypeptide analog to a native peptide or polypeptide of a protein comprising a Ser/Thr motive which is cleavable with a protease (e.g. trypsin protease). Said probe enables protein identification and absolute protein quantitation of a protein comprising a Ser/Thr motive with mass spectrometry, preferably with HPLC-MS or HPCL-MS/MS, particularly supported by multiple reaction monitoring (MRM). It preferably incorporates one stable isotope labeled amino acid, creating a slight increase (e.g. 6-10 daltons) in molecular weight. When mixed, the native peptide of the protein comprising a Ser/Thr motive and the synthetic peptide co-elute chromatographically, co-migrate electrophoretically, and ionize with the same intensity. Nevertheless, by mass spectrometry, the native peptide of the protein comprising a Ser/Thr motive and the synthetic peptide can easily be distinguished. In a typical mass spectrometry procedure, preferably HPLC-MS or HPLC-MS/MS procedure, a known amount of the synthetic peptide is added to a sample of a subject. The sample is then digested (e.g. by a protease such as trypsin protease) and analyzed by mass spectrometry, preferably by HPLC-MS or HPLC-MS/MS. Extracted ion chromatograms are generated for the native peptide of the protein comprising a Ser/Thr motive and the synthetic peptide internal standard. Using peak ratios, the quantity of the native peptide of the protein comprising a Ser/Thr motive is calculated. It also allows quantitation of the amount of the protein comprising a Ser/Thr motive in the sample of the subject. The techniques for selecting and preparing the synthetic peptide are well known in the art. For example, the skilled person knows how to select precursor-fragment-ion-transitions under the aspect of optimal selectivity and sensitivity. Preferably, the above mentioned protein comprising a Ser/Thr motive is Serpin A1. More preferably, the mass spectrometry probe (peptide) has an amino acid sequence according to SEQ ID NO: 5 to SEQ ID NO: 8 (see Table 2 in the examples). The mass spectrometry probe (peptide) may also have an amino acid sequence according to SEQ ID NO: 9 to SEQ ID NO: 23 (see Table 2 in the examples). It is preferred that the mass spectrometry probe (peptide) is a probe (peptide) which is able to recognize only a specific Serpin A1 isoform, e.g. a specific isoform which may be present in the biological sample from a subject.

In a third aspect, the present invention relates to means for (use in) the diagnosis and/or prognosis of Parkinson's disease dementia (PDD) or differential diagnosis and/or prognosis between Parkinson's disease (PD) and Parkinson's disease dementia (PDD) comprising or consisting of at least one molecule according to the second aspect.

It is preferred that said means allows the detection/determination of O-glycosylation in a protein comprising a Ser/Thr motive, e.g. Serpin A1, particularly the number of glycosylated isoforms of a protein comprising a Ser/Thr motive, e.g. Serpin A1 isoforms, and/or the level of sialic acid on a protein comprising a Ser/Thr motive, e.g. Serpin A1, preferably in a biological sample from a subject and, thus, the diagnosis and/or prognosis of PDD or the differential diagnosis and/or prognosis between PD and PDD.

With respect to the meaning and preferred embodiments of the terms “biological sample”, “protein comprising a Ser/Thr motive”, and “subject” it is referred to the definitions and explanations provided above. Also all other terms used in the description of this aspect have the meaning as described above.

Preferably, said means comprise a solid support. It is preferred that said means further comprises means for immobilising the at least one molecule according to the second aspect of the present invention on said solid support or for attaching the at least one molecule according to the second aspect of the present invention to said solid support. The solid support may be made of the following materials: glass (including modified or functionalized glass), plastics (including acrylics, polystyrene, polypropylene, polyethylene, polybutylene, polyurethanes, teflon, etc.), polysaccharides, nylon or nitrocellulose, resins, silica or silica-based materials (including silicon and modified silicon), carbon, metals or mixtures/combinations thereof. The solid support may be planar, e.g. a slide, chip, matrix, or array, although also other configurations of the solid support may be possible as well, e.g. tubes, beads, or microspheres.

It is particularly preferred that the at least one molecule according to the second aspect of the present invention is attached to or immobilized on the solid support.

It is more particularly preferred that said means consists of a solid support to which the at least one molecule according to the second aspect of the present invention is attached or on which the at least one molecule according to the second aspect of the invention is immobilized.

More preferably, said means comprise a biochip/microarray or a set of beads. It is preferred that said means further comprises means for immobilising the at least one molecule according to the second aspect of the present invention on the biochip or beads of said set of beads or for attaching the at least one molecule according to the second aspect of the present invention to the biochip or beads of said set of beads.

It is more particularly preferred that the at least one molecule according to the second aspect of the invention is attached to or immobilized on the biochip/microarray or that the at least one molecule according to the second aspect of the invention is attached to or immobilized on the beads of said set of beads.

It is most particularly preferred that said means consists of (i) a biochip to which the at least one molecule according to the second aspect of the present invention is attached or on which the at least one molecule according to the second aspect of the invention is immobilized, or (ii) beads of a set of beads to which the at least one molecule according to the second aspect of the present invention is attached or on which the at least one molecule according to the second aspect of the invention is immobilized.

It is preferred that said beads or microspheres have a mean diameter of between 2 to 20 microns, preferably 4 to 10 microns, most preferably 5 to 7 microns, i.e. 2, 3, 4, 5, 6, 7, 8, 9, 10, 11, 12, 13, 14, 15, 16, 17, 18, 19, or 20 microns.

The terms “biochip” and “microarray” are interchangeable used herein. The terms “attached” or “immobilized”, as used herein, refer to the binding between the molecule according to the second aspect of the present invention and the solid support, e.g. biochip, and may mean that the binding between the molecule according to the second aspect of the present invention and the solid support, e.g. biochip, is sufficient to be stable under conditions of binding, washing, analysis and removal. The binding may be covalent or non-covalent. Covalent bonds may be formed directly between the molecule according to the second aspect of the present invention and the solid support, e.g. biochip, or may be formed by a cross linker or by inclusion of specific reactive groups on either the solid support, e.g. biochip, or the molecule according to the second aspect of the invention, or both. Non-covalent binding may be electrostatic, hydrophilic and hydrophobic interactions or combinations thereof. Immobilization or attachment may also involve a combination of covalent and non-covalent interactions.

Preferably, the above-mentioned means comprises or consists of one or more molecule(s), e.g. 1, 2, 3, 4, 5, 6, 7, 8, 9, 10, 11, 12, 13, 14, 15, 16, 17, 18, 19, 20, or more molecule(s), according to the second aspect of the present invention. For example, the above-mentioned means may comprise or consists of a solid support to which the one or more molecule(s) according to the second aspect of the present invention is (arc) attached or on which the one or more molecule(s) according to the second aspect of the invention is (are) immobilized. Further, for example, the above-mentioned means may comprise or consists of (i) a biochip to which the one or more molecule(s) according to the second aspect of the present invention is (are) attached or on which the one or more molecule(s) according to the second aspect of the invention is (are) immobilized, or (ii) beads of a set of beads to which the one or more molecule(s) according to the second aspect of the present invention is (are) attached or on which the one or more molecule(s) according to the second aspect of the invention is (are) immobilized.

It is preferred that each of the above-mentioned molecules only binds to a specific protein comprising a Ser/Thr motive, e.g. Serpin A1. It is more preferred that each of the above-mentioned molecules only binds to a specific region of a specific protein comprising a Ser/Thr motive, e.g. Serpin A1, for example, a region comprised in the N-terminal and/or C-terminal region of the amino acid sequence of said protein, a region comprised in the glycan structure (including or excluding sialic acid) of said protein, or a sialic acid structure on said protein. It is most preferred that said molecule is an antibody. Thus, the above-mentioned means may comprise or consist of a solid support, preferably biochip or a set of beads, comprising the above-mentioned one or more molecule(s), particularly one or more antibodies.

It is further preferred that the molecule(s) according to the second aspect of the present invention or the means according to the third aspect of the present invention is (are) used in step (i) of the method of the first aspect of the present invention for detecting/determining O-glycosylation in a protein comprising a Ser/Thr motive, e.g. Serpin A1, particularly the number of glycosylated isoforms of a protein comprising a Ser/Thr motive, e.g. Serpin A1 isoforms, and/or the level of sialic acid on a protein comprising a Ser/Thr motive, e.g. Serpin A1, in a biological sample from a subject. The selection of the molecule(s) according to the second aspect of the present invention or the means according to the third aspect of the present invention depends on whether the O-glycosylation in a protein comprising a Ser/Thr motive, e.g. Serpin A1, particularly the number of glycosylated isoforms of a protein comprising a Ser/Thr motive, e.g. Serpin A1 isoforms, and/or the level of sialic acid on a protein comprising a Ser/Thr motive, e.g. Serpin A1, is (are) to be detected/determined in a biological sample from a subject (see first and second aspect of the present invention).

For example, in cases where the number of glycosylated isoforms of a protein comprising a Ser/Thr motive, e.g. Serpin A1 isoforms, is to be determined, it is preferred that the molecule, e.g. antibody, is able to bind to any glycosylated isoform, e.g. any Serpin A1 isoform, for example, to the amino acid sequence, such as N-terminal and/or C-terminal region, of said isoform, which may be present in the biological sample from a subject. It is more preferred that said molecule, preferably antibody, is able to bind to the N-terminal region between Glu25 to Leu200 or between Asp30 to Leu200, more preferably between Glu25 to Leu150 or between Asp30 to Leu150, even more preferred between Glu25 to Ile100 or between Asp30 to ILe 100, and most preferably between Glu25 to Ser60 or between Asp30 to Ser60, of the amino acid sequence according to SEQ ID NO: 1 to 3, or at an amino acid position corresponding thereto. Preferably, said molecule is used after performance of a 1D gel electrophoresis with isoelectric focusing (IEF) as first dimension, wherein the different glycosylated isoforms, e.g. Serpin A1 isoforms, are separated according to their isoelectric point (PI), or of a 2D gel electrophoresis with isoelectric focusing (IEF) as a first dimension and SDS-PAGE as a second dimension, wherein the different glycosylated isoforms, e.g. Serpin A1 isoforms, are separated according to their isoelectric point (PI) and their protein mass. The molecule, e.g. antibody, may also be able to bind only a specific glycosylated isoform, e.g. Serpin A1 isoform, which may be present the biological sample from a subject. For example, it may bind to a specific region of only a specific glycosylated isoform, e.g. Serpin A1 isoform, for example, a region comprised in the N-terminal and/or C-terminal region of the amino acid sequence of said isoform, a region comprised in the glycan structure (including or excluding sialic acid) of said isoform, or a sialic acid structure on said isoform. In this case, it may not be necessary to perform beforehand a 1D gel electrophoresis or of a 2D gel electrophoresis to hitherto separate the different glycosylated isoforms, e.g. Serpin A1 isoforms, for example, according to their isoelectric point (PI) and/or their protein mass. Further, for example, in cases where the number of glycosylated isoforms, e.g. Serpin A1 isoforms, is to be determined, it is preferred that the mass spectrometry probe is a probe which is able to recognize only a specific glycosylated isoform, e.g. Serpin A1 isoform, for example, a specific isoform which may be present in the biological sample from a subject.

Furthermore, for example, in cases where the level of sialic acid on a protein comprising a Ser/Thr motive, e.g. Serpin A1, is to be determined, it is preferred that the molecule, e.g. antibody, is able to hind to all sialic acid(s) which may be present on a protein comprising a Ser/Thr motive, e.g. Serpin A1, comprised in the biological sample from a subject. Said molecule, e.g. antibody, may be labeled with alkaline phosphatase or horseradish peroxidase which may catalyze chemiluminescent reactions (e.g. horseradish peroxidase) or colorimetric reactions (e.g. alkaline phosphatase). The level of sialic acid on a protein comprising a Ser/Thr motive, e.g. Serpin A1, may then be evaluated trough densitometry (how intense the stain/light is) or spectrophotometry. The detected level of sialic acid on a protein comprising a Ser/Thr motive, e.g. Serpin A1, may finally be compared with the level of sialic acid on a protein comprising a Ser/Thr motive, e.g. Serpin A1, of a control (see above) to carrying out diagnosing and/or prognosing. The molecule, e.g. antibody, may also be able to bind only a specific sialic structure on a protein comprising a Ser/Thr motive, e.g. Serpin A1, which may be present the biological sample from a test subject but which is usually not present in the biological sample from a control subject. If, for example, the specific sialic acid structure on a protein comprising a Ser/Thr motive, e.g. Serpin A1, isoforms can be detected, the subject tested is identified as experiencing PDD.

In addition, for example, the different glycosylated isoforms, Serpin A1 isoforms, comprised in a biological sample may be isolated from a biological sample by contacting said sample with a biochip or a set of beads to which the molecules, particularly antibodies, which specifically bind said glycosylated isoforms, e.g. Serpin A1 isoforms, are attached, wherein it is envisaged that specific binding of the glycosylated isoforms, e.g. Serpin A1 isoforms, comprised in said sample to said molecules occurs. Subsequently, said biochip or said set of beads is separated from said sample and optionally washed with a wash solution. Binding of the glycosylated isoforms, e.g. Serpin A1 isoforms, comprised in said sample may then be verified by a molecule, e.g. antibody, specific for any glycosylated isoform, e.g. Serpin A1 isoforms, and labeled with a detectable tag with chemiluminescence or colorimetric reactions. Finally, the number of the detected glycosylated isoforms, e.g. Serpin A1 isoforms, is determined and compared to a control (see above). If, for example, the number of the detected Serpin A1 isoforms exceeds 5, as 6 or 7 isoforms could be detected, the subject tested is identified as experiencing PDD.

It is preferred that the protein comprising a Ser/Thr motive is selected from the group consisting of a Serpin, preferably Serpin A1, Serpin A8, or Serpin F1; Fetuin A; Ceruloplasmin; Haptoglobin; and Zinc-alpha-2 glycoprotein. It is more preferred that the protein comprising a Ser/Thr motive is Serpin A1.

In a fourth aspect, the present invention relates to a kit for (use in) the diagnosis and/or prognosis of Parkinson's disease dementia (PDD) or for differential diagnosis and/or prognosis between Parkinson's disease (PD) and Parkinson's disease dementia (PDD) comprising
(i) a means for detecting O-glycosylation in a protein comprising a Ser/Thr motive and/or the level of sialic acid on said protein, and optionally
(ii) a data carrier, and/or
(iii) a container.

In the context of the present invention, a kit of parts (in short a kit) is understood to be any combination of at least some of the components identified herein, which are combined, coexisting spatially, to a functional unit, and which can contain further components.

It is preferred that the O-glycosylation in a protein comprising a Ser/Thr motive and/or the level of sialic acid on said protein is detected/determined in a biological sample from a subject. This may then allow the diagnosis and/or prognosis of PDD or the differential diagnosis and/or prognosis between PD and PDD.

With respect to the meaning and preferred embodiments of the terms “biological sample”, “protein comprising a Ser/Thr motive”, and “subject” it is referred to the definitions and explanations provided above. Also all other terms used in the description of this aspect have the meaning as described above.

It is further preferred that said means comprises or consists of
(i) at least one molecule according to the second aspect of the present invention,
(ii) a means according to the third aspect of the present invention,
(iii) a means for carrying out 1D gel electrophoresis, preferably means for isoelectric focusing (IEF), and optionally a sialidase enzyme, and/or
(iv) a means for carrying out 2D gel electrophoresis, and optionally a sialidase enzyme.

As to the terms “1D gel electrophoresis”, “isoelectric focusing (IEF)” or “2D gel electrophoresis” it is referred to the definitions mentioned above. The means for carrying out 1D gel electrophoresis particularly comprises or consists of a 1D gel such as a SDS, native, or IEF gel or the means for carrying out 2D gel electrophoresis particularly comprises or consists of a 2D gel such as a gel which allows separation of the proteins according to their isoelectric point and according to their protein mass.

A means for isoelectric focusing (IEF) or 2D gel electrophoresis preferably separates Serpin A1 isoforms, particularly differentially sialylated Serpin A1 isoforms, allowing the identification of the number of Serpin A1 isoforms occurring in subjects which may suffer from PDD and preferably also those occurring in control subjects. Examples of means for isoelectric focusing (IEF) are immobilized pH gradient (iPG) gels, iPG strips, preferably dry and rehydratable iPG strips, and microfluidic chip based isoelectric focusing (Sommer and Hatch, Electrophoresis. 2009 March; 30(5):742-5.).

The enzyme sialidase (also designated as neuroamidase) may also be comprised in said means. As already described above, the enzyme sialidase (also designated as neuroamidase) is able to digest terminal sialic, oligosialic and polysialic acids and can, thus, be used for the additional detection of a shift of differentially sialylated Ser/Thr motive comprising proteins, particularly Serpin A1, towards a more basic isoelectric point (pI) on a 1D gel with IEF as first dimension or 2D gel with IEF as second dimension. In case of Serpin A1, the disappearance of hypersialylated Serpin A1 isoforms, preferably the Serpin A1 isoforms with the lowest pI (e.g. with an pI in the range of 4.5 and 5), more preferably the one or two Serpin A1 isoforms with the lowest pI (e.g. with an pI in the range of 4.5 and 5), compared to a control sample (not treated with sialidase enzyme), is a sign for the presence of PDD (see examples, particularly FIG. 5).

As mentioned above, isoelectric focusing (IEF) allows the separation of proteins according to their isoelectric point (pI). In detail, a protein that is in a pH region below its isoelectric point (pI) will be positively charged and so will migrate towards the cathode. As it migrates through a gradient of increasing pH, however, the protein's overall charge will decrease until the protein reaches the pH region that corresponds to its pI (neutral stage, where the overall charge of the protein is 0). At this point it has no net charge and so migration ceases (as there is no electrical attraction towards either electrode). As a result, the proteins become focused into sharp stationary bands with each protein positioned at a point in the pH gradient corresponding to its pI. The technique of isoelectric focusing (IEF) is capable of extremely high resolution with proteins differing by a single charge being fractionated into separate bands.

If the enzyme sialidase is used, the following generally applies: Sialic acid leads to an acidification of the protein by giving it a more negative charge which in turn requires a more acidic pH to be neutralized in the IEF gel/strip. When the sialic acid residues are removed, the protein will be less negatively charged, thus, requiring a less acidic pH for neutralization. This leads to a shift to a more basic pI.

It is particularly preferred that the kits comprise materials desirable from a commercial and user standpoint such as a buffer(s), a reagent(s) and/or a diluent(s). Said materials may be useful for detecting/determining O-glycosylation in a protein comprising a Ser/Thr motive, e.g. Serpin A1, and/or the level of sialic acid on said protein, e.g. Serpin A1. It is further particularly preferred that the kits comprise reporter-means such as an affinity tag binding protein, for example, a biotin binding molecule (e.g. avidin or streptavidin) bound to a detectable label, e.g. an enzymatic, fluorescent or radioisotope label and/or a secondary antibody that is labeled with an affinity tag, for example, biotin. Said reporter-means may also be useful for detecting/determining O-glycosylation in a protein comprising a Ser/Thr motive, e.g. Serpin A1, and/or the level of sialic acid on said protein, e.g. Serpin A1.

The above-mentioned data carrier may be a graphically data carrier such as an information leaflet, an information sheet, a bar code or an access code, or an electronically data carrier such as a floppy disk, a compact disk (CD), or a digital versatile disk (DVD). The access code may allow the access to a database, e.g. an interne database, a centralized, or a decentralized database.

Preferably, said data carrier comprises a control (value), particularly to allow the interpretation of information obtained when performing the above-mentioned method for diagnosing and/or prognosing Parkinson's disease dementia (PDD) or differential diagnosing and/or prognosing between Parkinson's disease (PD) and Parkinson's disease dementia (PDD). Thus, said control (value) may allow for the diagnosis and/or prognosis of Parkinson's disease dementia (PDD) or for differential diagnosis and/or prognosis between Parkinson's disease (PD) and Parkinson's disease dementia (PDD). As to the definition of the control (value) and preferred embodiments of said control (value), it is referred to the first aspect of the present invention.

Additionally or alternatively, the data carrier comprises instructions for the method according to the first aspect of the present invention, the molecule according to the second aspect of the present invention and/or the means according to the third aspect of the present invention in order to diagnose and/or prognose Parkinson's disease dementia (PDD) or to differential diagnose and/or prognose between Parkinson's disease (PD) and Parkinson's disease dementia (PDD). Said data carrier may further comprise the following:
(i) instructions for use of the means for detecting/determining O-glycosylation in a protein comprising a Ser/motive, e.g. Serpin A1, and/or the level of sialic acid on said protein, e.g. Serpin A1, and/or instructions for use of the kit,
(ii) instructions with respect to the biological sample which is to be used and/or instructions how said biological sample is to be obtained from the subject,
(iii) quality information material such as information about the lot/batch number of the means for detecting/determining O-glycosylation in a protein comprising a Ser/Thr motive, e.g. Serpin A1, and/or the level of sialic acid on said protein, e.g. Serpin A1, and/or of the kit,
(iv) information concerning the detailed composition of the buffer(s), diluent(s) and reagent(s) useful for detecting/determining O-glycosylation in a protein comprising a Ser/Thr motive, e.g. Serpin A1, and/or the level of sialic acid on said protein, e.g. Serpin A1,
(v) warnings about possible miss-interpretations or wrong results when applying a wrong method and/or wrong means, and/or
(vi) warnings about possible miss-interpretations or wrong results when using wrong reagent(s) and/or buffer(s).

It is preferred that the protein comprising a Ser/Thr motive is selected from the group consisting of a Serpin, preferably Serpin A1, Serpin A8, or Serpin F1; Fetuin A; Ceruloplasmin; Haptoglobin; and Zinc-alpha-2 glycoprotein. It is more preferred that the protein comprising a Ser/Thr motive is Serpin A1.

Preferably, Serpin A1 has an amino acid sequence according to SEQ ID NO: 1 (Isoform 1), SEQ ID NO: 2 (Isoform 2) or SEQ ID NO: 3 (Isoform 3) (all human, see above), Serpin A8 has an amino acid sequence according to SEQ ID NO: 24 (human), Fetuin A has an amino acid sequence according to SEQ ID NO: 25 (human), Ceruloplasmin has an amino acid sequence according to SEQ ID NO: 26 (human), Serpin F1 has an amino acid sequence according to SEQ ID NO: 27 (human), Haptoglobin has an amino acid sequence according to SEQ ID NO: 28 (human), Zinc-alpha-2 glycoprotein has an amino acid sequence according to SEQ ID NO: 29 (human), or variants thereof (see above).

In a fifth aspect, the present invention relates to the use of the molecule(s) of the second aspect of the present invention, the means of the third aspect of the present invention, or the kit of the fourth aspect of the present invention in the method of the first aspect of the present invention, preferably in step (i) of said method. Accordingly, it is preferred that the molecule(s), the means, or the kit is (are) used to detect/determine the O-glycosylation in a protein comprising a Ser/Thr motive, e.g. Serpin A1, and/or the level of sialic acid on said protein, e.g. Serpin A1, in a biological sample from a subject. All terms used in the description of this aspect have the meaning as described above.

In a further aspect, the present invention relates to a molecule composition or set comprising at least two, e.g. 2, 3, 4, 5, 6, or 7, molecules for detecting O-linked glycomoieties in at least two, e.g. 2, 3, 4, 5, 6, or 7, proteins comprising a Ser/Thr motive and/or glycosylated isoforms of at least two, e.g. 2, 3, 4, 5, 6, or 7, Ser/Thr motive comprising proteins for (use in) the diagnosis and/or prognosis of Parkinson's disease dementia (PDD) or differential diagnosis and/or prognosis between Parkinson's disease (PD) and Parkinson's disease dementia (PDD). Said at least two proteins are preferably selected from the group consisting of a Serpin, preferably Serpin A1, Serpin A8, or Serpin F1; Fetuin A; Ceruloplasmin; Haptoglobin; and Zinc-alpha-2. Said molecule composition or set may alternatively be comprised in the means according to the third aspect of the present invention or kit according to the fourth aspect of the present invention. All terms used in the description of this aspect have the meaning as described above.

In another further aspect, the present invention relates to a molecule for detecting O-glycosylation in a protein comprising a Ser/Thr motive, e.g. Serpin A1, particularly the number of glycosylated isoforms of a protein comprising a Ser/Thr motive, e.g. Serpin A isoforms, and/or the level of sialic acid on said protein, e.g. Serpin A1, for (use in) the diagnosis and/or prognosis of Parkinson's disease dementia (PDD) or differential diagnosis and/or prognosis between Parkinson's disease (PD) and Parkinson's disease dementia (PDD). Said molecule may alternatively be comprised in the means according to the third aspect of the present invention or kit according to the fourth aspect of the present invention. All terms used in the description of this aspect have the meaning as described above.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE TABLES AND FIGURES

The following Tables are merely illustrative of the present invention and should not be construed to limit the scope of the invention as indicated by the appended claims in any way.

Table 1: Relevant parameters of all groups investigated. Data are indicated as mean±SD. Abbreviations: PD=Parkinson's disease, PDD=Parkinson's dementia, CON=control persons/subjects, m/f=male/female, MMST=minimal mental status test.

Table 2: 2D-DIGE analysis and identification of selected CSF proteins. Abbreviations: PD=Parkinson's disease, PDD=Parkinson's dementia, CON=control persons/subjects, pI=isoelectric point of the proteins, PMF=peptide mass fingerprint, MS=mass spectrometry.

Table 3: Posttranslational modifications of Serpin A1. Listing of glycosylation residues for Serpin A1 isoforms represented by spot 1 to 7 of a 2D DIGE experiment. Abbreviations: HexNAc=N-acetyl-hexosamine, Hex=hexose (mannose, glucose or galactose), NeuAc=sialic acid.

The following Figures are merely illustrative of the present invention and should not be construed to limit the scope of the invention as indicated by the appended claims in any way.

FIG. 1: Representative 2D-DIGE gel of CyDye-labeled CSF-proteins. Indication is given in black-white manner for better visibility. Arrows indicate identified protein spots: number 1 for Serpin A1+Serpin A8, number 2 for Serpin A1, number 3 for Fetuin A, number 4 for Ceruloplasmin, number 5 for Serpin F1, number 6 for Haptoglobin and number 7 for Zinc-alpha-2 glycoprotein (numbers correlate with the numbers in the first row of Table 2). The magnification shows spot number 2 (Serpin A1) in CyDye overlay. Abbreviations: pI=isoelectric point of the proteins; MW=molecular weight in kilo-Dalton.

FIG. 2: Identification and regulation of Serpin A1 and its different isoforms. 2A illustrates the 2D DICE analysis with the pixel volume distribution and regulation for Serpin A1 corresponding to number 2 in FIG. 1. 2B shows the isoform distribution of Serpin A1 of a representative PDD gel with spectral counts for the respective isoforms.

FIG. 3: 1D and 2D Immunoblots of Serpin A1. 3A shows 1D immunoblot band volumes of Westernblots (adjusted for membrane background) of Serpin A1. 3B indicated the statistical analysis for Serpin A1 indicated in the 2D DIGE experiment with a significant regulation of said Serpin A1. 3C illustrates the 2D immunoblot of Serpin A1 with the different spot-pattern in PD/CON and PDD with the relevant additional spots 1 and/or 2 in PDD. 3D and 3E show the distribution of spot pattern in the groups with sensitivity and specificity for the differentiation of PDD versus PD. Abbreviations: PD=Parkinson's disease, PDD=Parkinson's dementia, CON=control persons, pI=isoelectric point of the proteins (3D: with Serpin A1 spots≦5 and >5, and 3E: with Serpin A1 spots≦5 and ≧6 spots (more stringent evaluation, ROC-analysis)).

FIG. 4: Immunoblots of Serpin A1 in human cortex tissue. 4A shows 1D westernblot in two CON and two patients with Lewy body dementia as a pathophysiological correlate of Parkinson's dementia. The protein can be identified in both tissues of control persons and diseased patients. 4B illustrates 2D westernblot for Serpin A1 of the patients investigated in 4A. The isoform pattern seen in CSF of CON/PD and PDD (FIG. 3C) with spot 1 and/or 2 indicative for PDD could not be reproduced in human cortex tissue. Abbreviations: CON=control persons, DLB=Lewy body dementia, pI=isoelectrie point of the proteins.

FIG. 5: Investigation of posttranslational modifications. Immunoblot of Serpin A1 in a PDD-patient with and without neuraminidase-treatment (different exposures of times are shown to better visualize the individual spots, intensity of both blots were similar; exposure time for the immunoblot without neuraminidase treatment=2 seconds, exposure time for the immunoblot with neuraminidase treatment=10 seconds). Treatment with the enzyme not only leads to an isoform shift towards a more basic pI but also to the disappearance of the diagnostic relevant (most acidic) spots 1 and/or 2. “Untreated” means usage of a native CSF-sample without neuraminidase digest.

FIG. 6: Representative Serpin A1-blots of PNGase F-treated CSF of PD and PDD. Treatment with PNGase F leads to a size-shift but not to an isoelectric point (pI)-shift in CSF of PD and PDD compared to a control (not treated with PNGase F). Indication of size in kDa.

FIG. 7: Multiple sequence alignment—Serpin A1 isoforms 1 (SEQ ID NO: 1), 2 (SEQ ID NO: 2), and 3 (SEQ ID NO: 3), Homo sapiens (human).

### EXAMPLES

The examples given below are for illustrative purposes only and do not limit the invention described above in any way.

### Example 1

### PDD Patients can be Identified on the Basis of Glycosylated Isoforms Such as Serpin A1 Isoforms

### Methods

### Subjects

All CSF samples used for the proteomic approach were taken from patients attending the general outpatient clinic (University of Ulm, Department of Neurology) in 2006 and 2007. CSF was stored at −80° C. after analysis of the routine parameters cell count, lactate, Q-albumin and total protein until further analysis. For the validation study, additional samples to the CSF-patients were obtained in blinded manner from two different centres: Department of Neurology, Kuopio, Finland (9 PD, 7 PDD) and Department of Neurology, Perugia, Italy (8 PD, 8 PDD). Collection and analysis of CSF samples was approved by the Ethics Committees and conformed to the requirements of the declaration of Helsinki in 1964. Particularly, the local ethics committees (Ethik-Kommission der Medizinischen Fakultät der Universitäten Ulm und Göttingen, approval numbers: 8801 and 100305 and the regional Ethical Committee Board (CEAS) of the University of Perugia, protocol number 19369/08/AV as well as the Ethics Committee of Kuopio University Hospital, number 5/2002) approved all experiments within our study.

All individuals or in case of demented patients their relatives gave written informed consent to their participation in the study and underwent a clinical, neurological, and neuroradiological examination as well as a short neuropsychological screening to investigate global cognitive functioning. Patients were examined neuropsychologically for unambiguous classification of their mental status and exclusion of depressive syndroms. All PD and PDD patients in Ulm were investigated with a detailed psychometric test battery (Bothe, M. R., Uttner, I., Otto, M., Sharpening the boundaries of Parkinson-associated dementia: recommendation for a neuropsychological diagnostic procedure. Journal of Neural Transmission., 2010. 117: p. 353-67) covering the following tests:

MMSE (Anthony, J. C., et al., Limits of the ‘Mini-Mental State’ as a screening test for dementia and delirium among hospital patients. Psychol Med, 1982. 12(2): p. 397-408), Geriatric Depression Scale (Yesavage, J. A., et al., Development and validation of a geriatric depression screening scale: a preliminary report. J Psychiatr Res, 1982. 17(1): p. 37-49), Parkinson Neuropsychometric Dementia Assessment (Kalbe, E., et al., Screening for cognitive deficits in Parkinson's disease with the Parkinson neuropsychometric dementia assessment (PANDA) instrument. Parkinsonism Relat Disord, 2008. 14(2): p. 93-101), Regensburger Wortflüssigkeitstest (RWT) (Tucha, O., S. Aschenbrenner, and K. W. Lange, Mirror writing and handedness. Brain Lang, 2000. 73(3): p. 432-41), Doors Test (Sunderland, A., et al., Subjective memory assessment and test performance in elderly adults. J Gerontol, 1986. 41(3): p. 376-84), Alertness/Go/Nogo/geteilte Aufinerksamkeit (Fimm, B., et al., Different mechanisms underly shifting set on external and internal cues in Parkinson's disease. Brain Cogn, 1994. 25(2): p. 287-304), Boston Naming Test (Mohs, R. C., W. G. Rosen, and K. L. Davis, The Alzheimer's disease assessment scale: an instrument for assessing treatment efficacy. Psychopharmacol Bull, 1983. 19(3): p. 448-50), Wechsler-Memory Scale (WMS-R) (Sullivan, K., Estimates of interrater reliability for the Logical Memory subtest of the Wechsler Memory Scale-Revised. J Clin Exp Neuropsychol, 1996. 18(5): p. 707-12), Verbaler Lern-und Merkfähigkeitstest: Helmstaedter, Coloured Progressive Matrices (Orme, J. E., The Coloured Progressive Matrices as a measure of intellectual subnormality. Br J Med Psychol, 1961. 34: p. 291-2), VOSP (Rapport, L. J., S. R. Millis, and P. J. Bonello, Validation of the Warrington theory of visual processing and the Visual Object and Space Perception Battery. J Clin Exp Neuropsychol, 1998. 20(2): p. 211-20), Uhrentest (Rapport, L. J., S. R. Millis, and P. J. Bonello, Validation of the Warrington theory of visual processing and the Visual Object and Space Perception Battery. J Clin Exp Neuropsychol, 1998. 20(2): p. 211-20), Wortschatztest: Schmidt, Metzler.

The number of patients per group, tau protein values, the number of Serpin A1 spots in the subgroups, the minimental status test (MMST) for all groups as well as Hoehn&Yahr stages for PD and PDD are indicated in Table 1. Tau protein and beta-amyloid levels were measured in each clinical centre with different cut-offs for β-amyloid so that further calculations were performed with tau protein levels only.

The diagnosis of all PD and PDD patients was made in accordance with the consensus criteria for PD/PDD (McKeith, I. G., et al., Consensus guidelines for the clinical and pathologic diagnosis of dementia with Lewy bodies (DLB): report of the consortium on DLB international workshop. Neurology, 1996. 47(5): p. 1113-24) as well as on the basis of the DSM-IV criteria and was established by neurologists in cooperation with neuro-psychologists, both blinded with regard to the neurochemical outcome measures.

### Control Subjects (CON)

The control patients showed neither extrapyramidal-motor nor dementia-specific symptoms. The final diagnoses of the patients were as follows: vertigo (n=1), paresthesia (n=2), ischemia (n=6), complex focal seizures (n=3), pseudotumor cerebri (n=1), lumbo-ischialgia (n=1), migraine (n=1), sharp-syndrom (n=1), polyradiculopathy (n=1) and dissociative disorders (n=1). For further data see Table 1.

### Neuropathology

Samples of human brain cortex tissue from 2 patients with Lewy body dementia (DLB) (age of 63 and 80 years, tau pathology of Braak stage II and III, Lewy-bodies neocortically circumscribed) and 2 CON (age of 59 and 46 years, tau pathology of Braak stage 0 and I, no Lewy-bodies) were obtained from the German Brain Bank (Ludwig-Maximilians University, Munich).

### CyDye Labeling

Proteomic analysis via 2D-DIGE was done with a volume-based normalization as described previously (Brechlin, P., et al., Cerebrospinal fluid-optimized two-dimensional difference gel electrophoresis (2-D DIGE) facilitates the differential diagnosis of Creutzfeldt-Jakob disease. Proteomics, 2008. 8(20): p. 4357-66) with the exception that 6 individual samples of each group were compared. In brief, 400 μl of each CSF sample were concentrated by VivaSpin columns with a 3 kDa cut-off (Sartorius Biolabs products), then albumin and immunglobuline-depleted. For subsequent conventional gel staining, the depleted CSF was acetone-precipitated and resuspended in 7 M urea, 2 M thiourea, 4% CHAPS, 1% DTT, % IPG Buffer (40%) pH 4-7 by rocking for 1 h at ambient temperature procedures. For CyDye labeling, precipitated proteins were lysed in 7 M Urea, 2 M Thiourea, 4% CHAPS, 30 mM Tris-HCl pH 8.1 at 10° C. Insoluble fractions were removed by centrifugation. For CSF proteome comparison in the first instance, six individual CSF samples of each group were compared by the mixed internal standard methodology described by Alban et al. (Alban, A., et al., A novel experimental design for comparative two-dimensional gel analysis: two-dimensional difference gel electrophoresis incorporating a pooled internal standard. Proteomics, 2003. 3(1): p. 36-44). CSF proteins were labeled with CyDyes™ (GE Healthcare), fluorescent dyes developed for the difference gel electrophoresis-system (2D-DIGE). Individual samples were labeled either with Cy3 or Cy5 for a dye-switched comparison to avoid potential dye-to-protein preferences. For the mixed internal standard, aliquots of each individual sample (corresponding to 100 ml CSF) included in the experiment were pooled and labeled with Cy2 in the same dye-to-CSF ratio. The labeling reaction was stopped by adding 20 nmol lysine. The labeled samples were combined and diluted 1.33× by a stock solution containing 7 M urea, 2 M thiourea, 4% CHAPS, 4% IPG-buffer pH 4-7, 4% DTT w/v for subsequent IEF.

### 2D Gel Electrophoresis and Imaging

isoelectric focusing was also done as described previously (Brechlin, P., et al., Cerebrospinal fluid-optimized two-dimensional difference gel electrophoresis (2-D DIGE) facilitates the differential diagnosis of Creutzfeldt-Jakob disease. Proteomics, 2008. 8(20): p. 4357-66). Second dimension SDS-PAGE was performed with homogeneous 12.5% gels (254×200 mm) according to Tastet et al. (Tastet, C., et al., A versatile electrophoresis system for the analysis of high-and low-molecular-weight proteins. Electrophoresis, 2003. 24(11): p. 1787-94) at 3.5 W/gel overnight at 20° C. The fluorescence signals of the three differently Cy-labeled protein samples were imaged using a laser scanner (DIGE-imager GE Healthcare) recording emission wavelengths of 520 nm (Cy2), 580 nm (Cy3) and 670 nm (Cy5) respectively. Proteins were post-stained with silver. Spots of interest were excised manually and subjected to mass spectrometric protein identification.

### In-Gel Digest, Mass Spectrometry and Database Search

Manually excised gel plugs were subjected to an automated platform for the identification of gel-separated proteins (Jahn, O., et al., Technical innovations for the automated identification of gel-separated proteins by MALDI-TOF mass spectrometry. Anal Bioanal Chem, 2006. 386(1): p. 92-103) as described in recent DIGE-based (Hu, Y., et al., Comparative proteomic analysis of intra-and interindividual variation in human cerebrospinal fluid. Mol Cell Proteomics, 2005. 4(12): p. 2000-9; Steinacker, P., et al., Unchanged survival rates of 14-3-3gamma knockout mice after inoculation with pathological prion protein. Mol Cell Biol, 2005. 25(4): p. 1339-46; Werner, H. B., et al., Proteolipid protein is required for transport of sirtuin 2 into CNS myelin. J Neurosci, 2007. 27(29): p. 7717-30; Brechlin, P., et al., Cerebrospinal fluid-optimized two-dimensional difference gel electrophoresis (2-D DIGE) facilitates the differential diagnosis of Creutzfeldt-Jakob disease. Proteomics, 2008. 8(20): p. 4357-66; Jesse, S., et al., A proteomic approach for the diagnosis of bacterial meningitis. PLoS One. 5(4): p. e10079) and large-scale proteome studies (Jahn, O., et al., Technical innovations for the automated identification of gel-separated proteins by MALDI-TOF mass spectrometry. Anal Bioanal Chem, 2006. 386(1): p. 92-103; Reumann, S., et al., Proteome analysis of Arabidopsis leaf peroxisomes reveals novel targeting peptides, metabolic pathways, and defense mechanisms. Plant Cell, 2007. 19(10): P. 3170-93). Briefly, a peptide mass fingerprint (PMF) and six fragment ion spectra for each sample were recorded automatically with an Ultraflex MALDI-ToF mass spectrometer (Broker Daltonics) under the control of the FlexControl 3.0 operation software. Post-processing of mass spectra and generation of peak lists was performed with the FlexAnalysis 3.0 software (Bruker Daltonics).

PMF and MS/MS data sets were batch-processed using the BioTools 3.1 software (Bruker Daltonics) as interface to the Mascot 2.2 software (Matrix Science) licensed in-house. Database searches were performed in the Swiss-Prot primary sequence database, restricted to the taxonomy homo sapiens. Carboxamidomethylation of Cys was specified as fixed and oxidation of Met as variable modification. One trypsin missed cleavage was allowed. Mass tolerances were set to 100 ppm for PMF searches and to 100 ppm (precursor ions) and 0.7 Da (fragment ions) for MS/MS ion searches. The minimal requirement for accepting a protein as identified was at least one peptide sequence match above identity threshold in coincidence with at least 20% sequence coverage assigned in the PMF.

### Westernblot

Equal amounts of total protein or equal volumes (CSF) were denatured and subjected to a SDS-PAGE in 12% polyacrylamide gels. Proteins were transferred onto PVDF membranes (Millipore, USA) and correct transfer was checked by Ponceau Red S staining. The membranes were incubated with the respective primary antibody (see below) followed by incubation with appropriate HRP conjugated secondary antibodies. Signal detection was performed by enhanced chemiluminescence (GE healthcare) on a CCD-camera. For 2D-Westernblotting, strips were equilibrated in 2×20 min in 6 M urea, 125 mM Tris-HCL pH 7.85, 3% SDS and 20% glycerol (v/v). 1% dithiothreitol (DTT) and 4.2% iodoacetic acid (IAA) were added for the first and second equilibration step respectively. The following primary antibodies were used: Ceruloplasmin (BD-Biosciences 611488), Fetuin A (R&D Systems BAF1184), Haptoglobin Hp2 (Abeam AB52652), Serpins A1, A8, F1 (R&D Systems MAB1268, BAF3156, BAF 1177) and Zinc-alpha-2 Glycoprotein (BD Biosciences 612354).

### Calculations and Statistics

Band volumes of Westernblots (adjusted for membrane background) were determined using the Quantity One software (BioRad). Analysis for significant differences in a given parameter between all tested groups or between two groups were calculated by Kruskal-Wallis test or Mann-Whitney test, respectively (sigma stat software). Correlation between parameters was examined applying Spearman rank correlation. P-values below p=0.05 were considered to be significant. For ROC-analysis, p-values p≦0.01 were considered significant (sigma plot software 10.0). Standard measures of diagnostic test validity such as sensitivity and specificity were calculated for the diagnostic groups (Altman, D. G. and J. M. Bland, Diagnostic tests. 1: Sensitivity and specificity. BMJ, 1994. 308(6943): p. 1552).

### Results

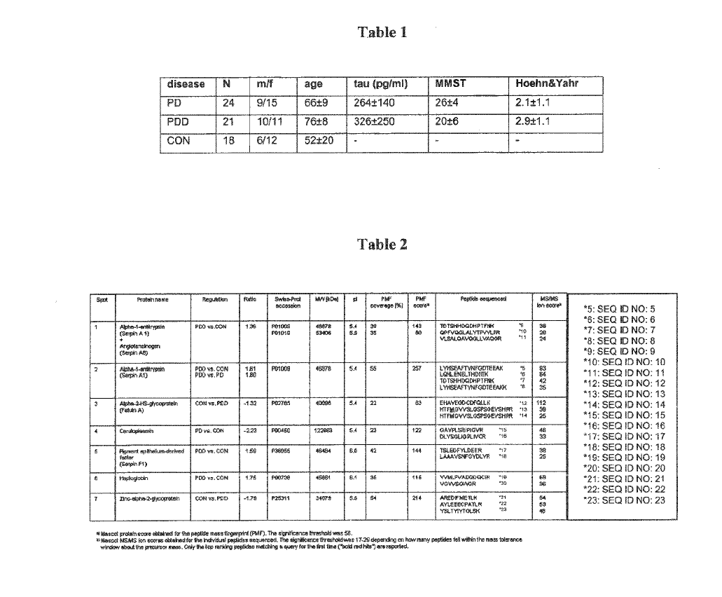

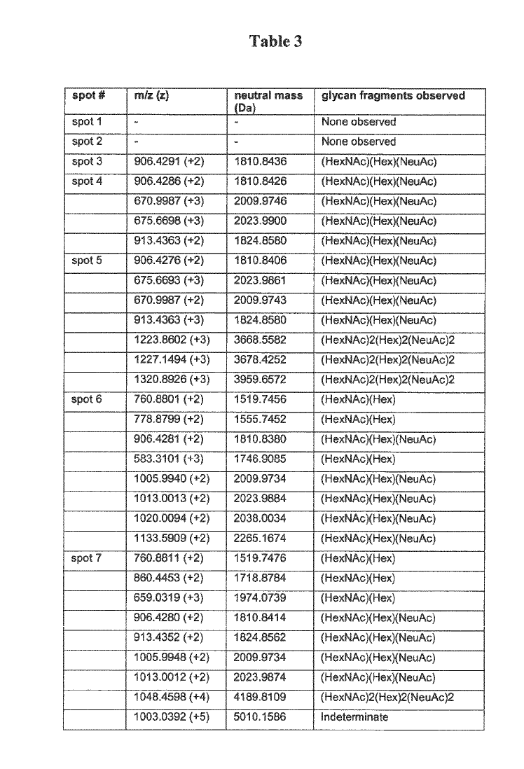

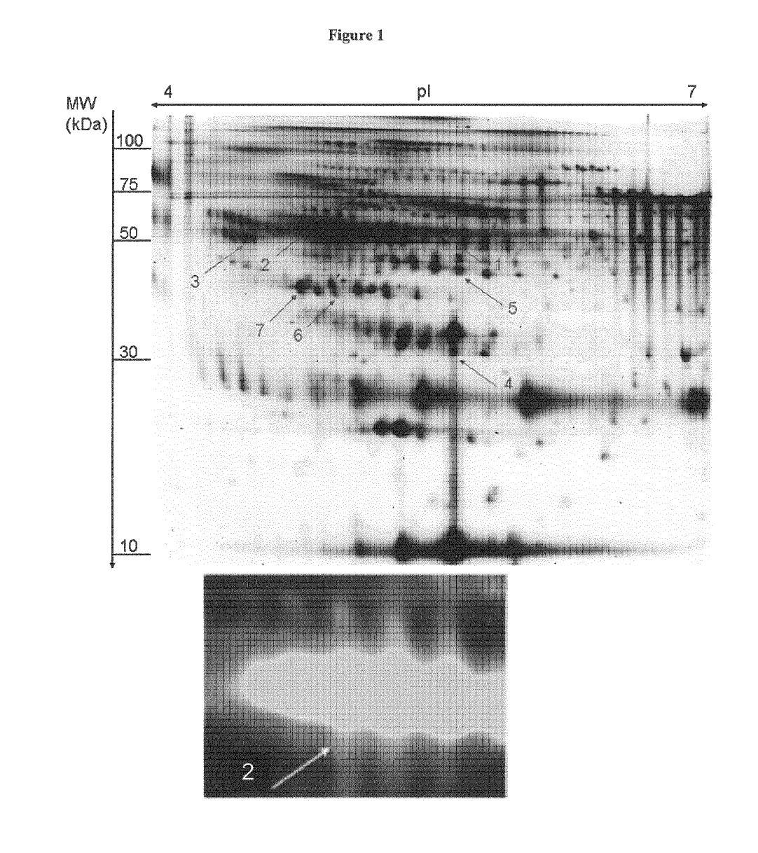

In the first step, identification of regulated proteins relevant for differentiation of PD and PDD was examined by means of 2D-DIGE experiments. CSF samples of 6 patients per group (PD, PDD and CON) were analysed, whereby an internal standard consisting of a mixture of all 18 samples was used to guarantee the comparability of the gels during the subsequent software-based evaluation. Samples were not pooled but 2 patients of different groups were loaded onto a gel together with the internal standard so that altogether 18 gels were analysed. Also a dye-switch was made to exclude false results due to preferential binding of certain proteins to one dye. A representative gel is shown in FIG. 1. Relevant regulated proteins were identified using MALDI-TOF MS/MS analysis. Spot data for the identified proteins are shown in Table 2. The characteristics of all patients in the study are given in Table 1.

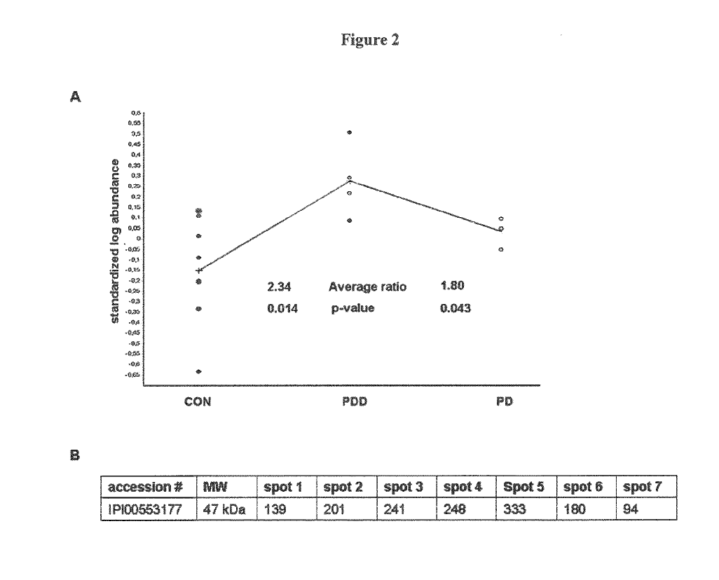

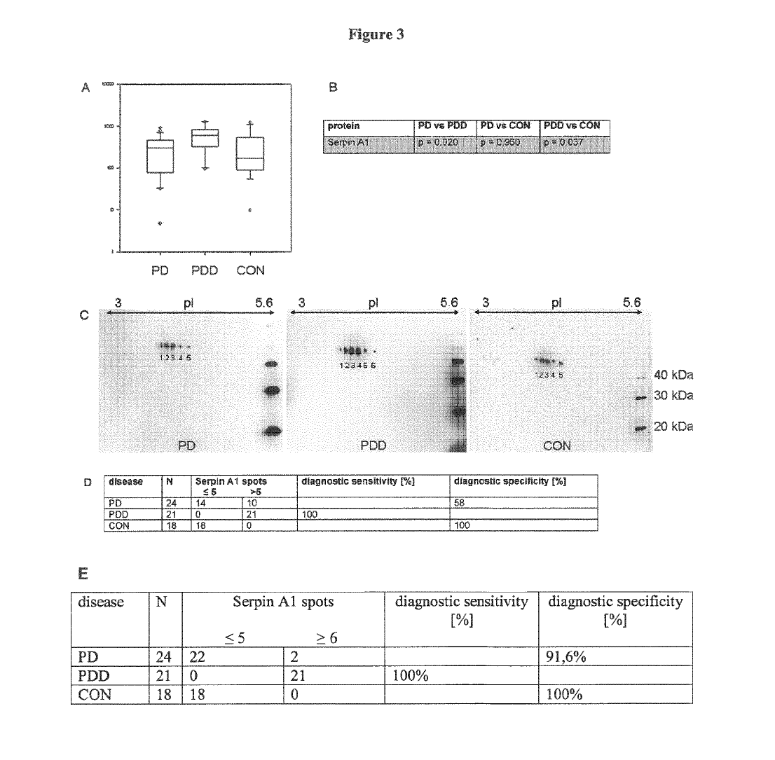

In a second step, the reproducibility of the 2D DIGE-data was examined using protein-biochemical methods like western blotting. In order to maintain comparability with the proteomic 2D-DIGE, samples were also used volume-normalized. After quantitative analysis of the protein-bands, Serpin A1 showed a statistically significant regulation between PDD on one side and PD/CON on the other (see FIG. 2A) with large overlap between the analysed groups (see FIG. 3A/B).

In addition, 2D immunoblots for Serpin A1 were performed to identify possible isoforms of this protein in the different groups. Here indeed, a different isoform-pattern was detected with ≦5 spots in the PD and CON groups and 6 or more spots in the PDD group. Spots indicated as spot 1 and spot 2 are additionally seen in the PDD patients (see FIG. 3C). These results could also be reproduced in the CSF-samples from Kuopio/Finland and Perugia/Italy, samples which were investigated in a blinded manner to test reproducibility of the data and to exclude a centre effect caused by pre-analytical handling procedures of the CSF-samples.

In a next step, the sensitivity as well as the specificity of Serpin A1 regarding its relevance as a possible diagnostic marker was examined to differentiate between PD and PDD. For all patients with the diagnosis of PDD, a diagnostic sensitivity of 100% was reached by the 2D immunoblot approach. In addition, a diagnostic specificity of 58% was reached for PD. In the relevant diagnostic PD group, the additional spots were seen in 10 out of 24 patients; interestingly, two of these patients developed a dementia in the course of disease (Table in FIG. 3D).

The Table in FIG. 3E shows a more stringent evaluation. For this, the cut-off of 5.5 spots using ROC-analysis was analysed (sensitivity=100%, CI 0.6915 to 1.000; specificity 100%, CI 0.7684 to 1.000). Using this cut-off, PD and PDD were compared and a diagnostic sensitivity of 91.6% and a specificity of 100% by 2D immunoblot approach were found. In the relevant diagnostic PD group, the additional spots were seen in 2 out of 24 patients; interestingly, these two patients developed a dementia in the course of disease.

### Discussion

Parkinson's dementia (PDD) is diagnosed according to clinical criteria and neuropsychological examinations (Truong, D. D. and E. C. Wolters, Recognition and management of Parkinson's disease during the premotor (prodromal) phase. Expert Rev Neurother, 2009. 9(6): p. 847-57). Since the typical Parkinson symptoms are initially predominant, the cognitive impairments or even a dementia is often neglected or delayed detected in advanced stages (Dubois, B. and B. Pillon, Cognitive deficits in Parkinson's disease. J Neurol, 1997. 244(1): p. 2-8; Poewe, W., et al., Diagnosis and management of Parkinson's disease dementia. Int J Clin Pract, 2008. 62(10): p. 1581-7). In order to identify PD patients who are at risk to develop a dementia, a laboratory marker was of great advantage.

In this example, CSF-analysis of patients with PD and PDD was performed using proteomic methods in order to detect proteins of potential diagnostic value. The serine-protease-inhibitor Serpin A1 could be verified with biochemical methods to be statistically significant regulated—a protein that was already described to be relevant in Alzheimer's disease and dementia with Lewy-bodies (Nielsen, H. M., et al., Plasma and CSF serpins in Alzheimer disease and dementia with Lewy bodies. Neurology, 2007. 69(16): p. 1569-79). In addition, the isoform-distribution of Serpin A1 was characterized and a different protein-pattern with 6 or more spots in PDD and with 5 spots or less in PD and CON was identified, wherein spots 1 and/or 2 were indicative for differentiation with 100% sensitivity for PDD. These results could be verified in a larger cohort of patients of three different specialized centres (Ulm/Germany, Perugia/Italy and Kuopio/Finland) whereby two PD cases presented the 6 spot-pattern which interestingly developed a dementia in the course of their disease. Thus, the isoform-pattern does not only help to support the diagnosis of PDD but has also a predictive value.

### Example 2

### Analysis of Posttranslational Modifications of the Serpin A1 Isoforms

### Methods

Characterization of Serpin A1 isoforms LC-MS/MS

Samples were subjected to proteolytic digestion on a ProGest (Genomic Solutions) workstation as follows: Samples were reduced with DTT at 60° C. and then allowed to cool to room temperature. Furthermore, samples were alkylated with iodoacetamide and subsequently incubated at 37° C. for 4 h in the presence of trypsin. Formic acid was added to stop the reaction and the supernatant was analyzed directly.

The samples were analyzed by nano LC/MS/MS on a ThermoFisher LTQ Orbitrap XL. 30 μl of hydrolysate was loaded onto a 5 mm×75 μm ID C12 (Jupiter Proteo, Phenomenex) vented column at a flow-rate of 10 μl/min. Gradient elution was over a 15 cm×75 μm ID C12 column at 300 nl/min. A 30 min gradient was employed. The mass spectrometer was operated in data-dependent mode and the six most abundant ions were selected for MS/MS. The Orbitrap MS scan was performed at 60,000 FWHM resolutions. MS/MS data were searched using a local copy of Mascot (www.matrixscience.com). The parameters for all LC/MS/MS searches were as follows: Type of search: MS/MS ion search. Taxonomy: human. Enzyme: trypsin. Fixed modifications: carbamidomethyl (C). Variable modifications: oxidation (M), acetyl (N-term), pyro-glu (N-term Q), methyl (various), deamidation (NQ), PO4 (STY). Mass values: monoisotopic. Protein mass: unrestricted. Peptide mass tolerance: ±10 ppm (Orbitrap). Fragment mass tolerance: ±0.5 Da (LTQ). Maximum missed cleavages: 2.

### PNGase F and Neuraminidase Digests

In order to assess possible glycosylations or sialylations of the serpin A1 isoforms, 5 μl of CSF was digested with PNGase F (New England Biolabs) or neuraminidase (Roche, 1585886) as stated by the manufacturers and subjected to a 2D-immunoblot. PNGase F is able to remove N-linked glycans and neuraminidase is able to remove terminal sialylation from N- and O-linked glycans.

### Neuraminidase Assay

To quantitatively assess the neuraminidase level in the CSF, a commercially available Neuraminidase Assay kit (Molecular probes) was used according to the manufacturer's instructions. CSF was measured in a 1+1 dilution.

Other methods were carried out as described in Example 1.

### Results

To further characterize the additional Serpin A1 spots, a mass-spectrometric analysis of the isoforms detected in the immunoblots was done by LC-MS/MS using a LTQ Orbitrap XL mass-spectrometer. Here, Serpin A1 was detected in all 7 spots from a representative gel of a PDD-patient being the dominant protein in spots 1 through 5. Serpin A1 was also detected in spots 6 and 7 but the dominant protein was identified as GC vitamin D-binding protein precursor (see FIG. 2B).

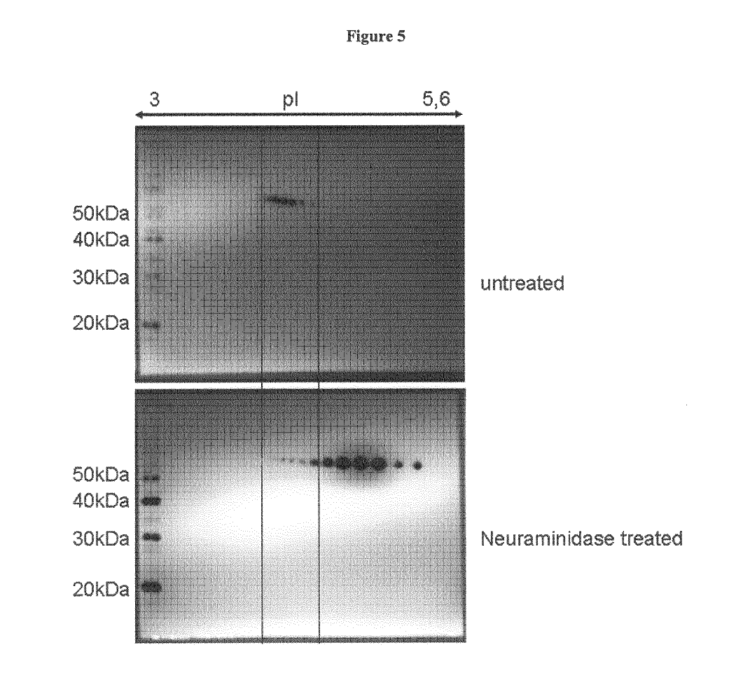

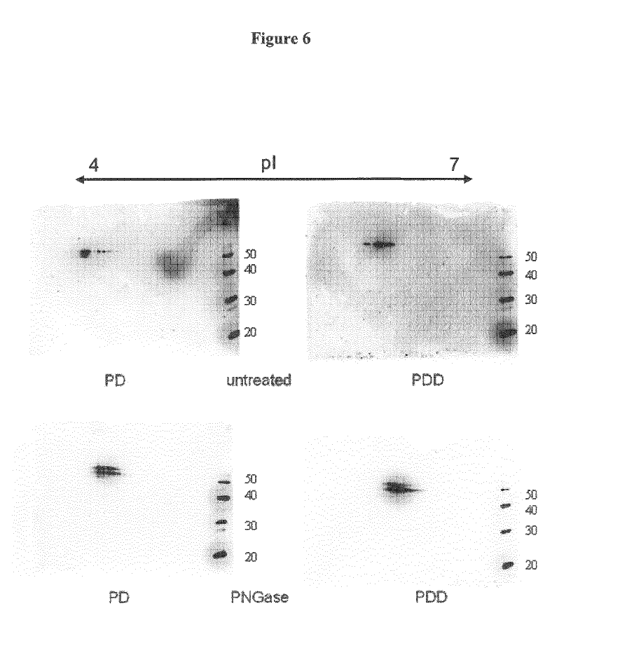

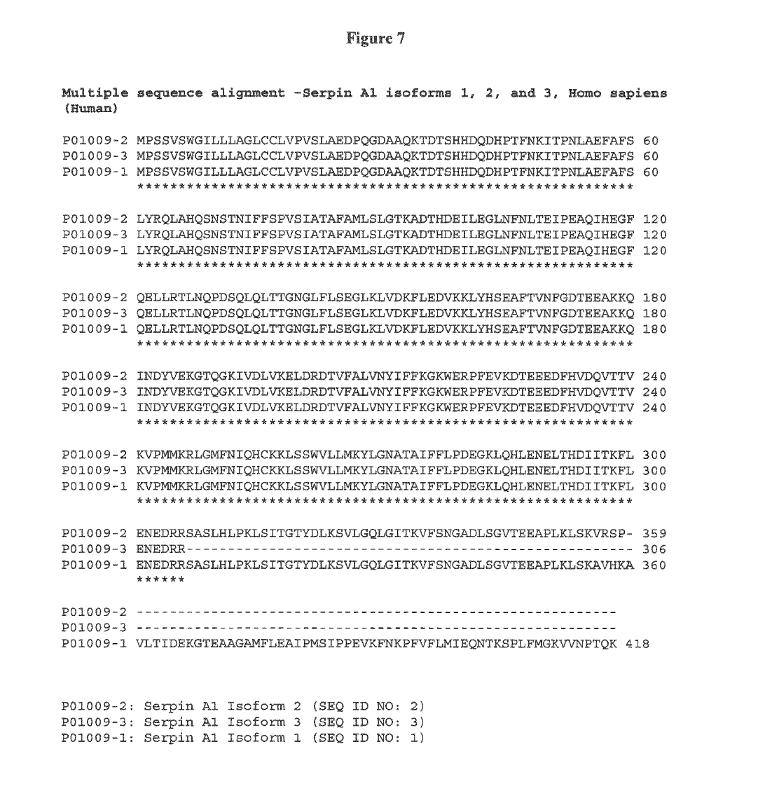

Posttranslational modifications with search for possible glycosylations and phosphorylations were performed for the Serpin A1 isoforms that failed to identify phosphorylations in any of the Serpin A1-spots. Glycosylations were detected for spots 3 to 7 but not for spots 1 and 2 (see Table 3) which are the diagnostic relevant ones to differentiate between PD and PDD. As this does not necessarily mean that there are no glycosylations in spots 1 and 2, a PNGase F digest was performed that detected all Serpin A1 spots in a PDD-patient to harbour N-glycosylations (see FIG. 6). However, as the additional Serpin A1 spots are still present after PNGase F treatment, N-linked glycans (or more precisely their terminal sialic acids) cannot be responsible for the altered charge states. It was therefore hypothesized that sialylated O-linked glycans may be the underlying posttranslational modifications for the characteristic Serpin A1 spot pattern. This hypothesis was tested by performing a neuraminidase-digest. A shift of the Serpin A1 isoforms towards a more basic pI (see FIG. 5) was indeed found. Most importantly, the diagnostic relevant acidic spots disappeared, indicating a hypersialylation of those isoforms.

After Neuraminidase digest, the spot pattern was similar in all groups, indicating sialylation as the cause for the different spot patt found in PDD patients. PNGase F digest of CON patients yielded only a size shift of the 5 spots present before digest but also no shift in pI.

This hypersialylation is not due to a decrease of neuraminidase (enzyme responsible for desialylation) in CSF, since its activity in CSF remains unchanged (data not shown in detail). Only briefly, the neuraminidase activity was determined using a fluorescence assay. The activity is indicated in arbitrary units: Mean values were CON: 22000; PD: 23000; and PDD: 22500.

### Discussion

Further analyses looking for posttranslational modifications were performed with evidence for glycosylations of the Serpin A1-isoforms. As glycosylation processes can be identified on N-linked or on O-linked glycans, both enzymes responsible for this, PNGase F and neuraminidase, were tested. PNGase F is able to remove N-linked glycans and neuraminidase is able to remove terminal sialylation from N- and O-linked glycans. Only treatment with the latter resulted not only in a shift of the Serpin A1 isoforms towards a more basic pI but also in the disappearance of the two relevant (most) acidic spots in PDD, indicating an O-linked hypersialylation of those isoforms (by neuraminidase) to be responsible for altered charged states. Thereby, hypersialylation does not seem to be traced back to a decrease in sialidase-activity (neuraminidase), as activity of this enzyme does not change in the CSF of any of the groups analysed.

Thus, it can be concluded that the sialylated, particularly hypersialylated, isoforms of Serpin A1 have a predictive value for the development of a dementia in PD patients.

### Example 3

### Source of Additional Serpin A1 is the Brain

### Methods

Methods were carried out as described in Examples 1 and 2.

### Results

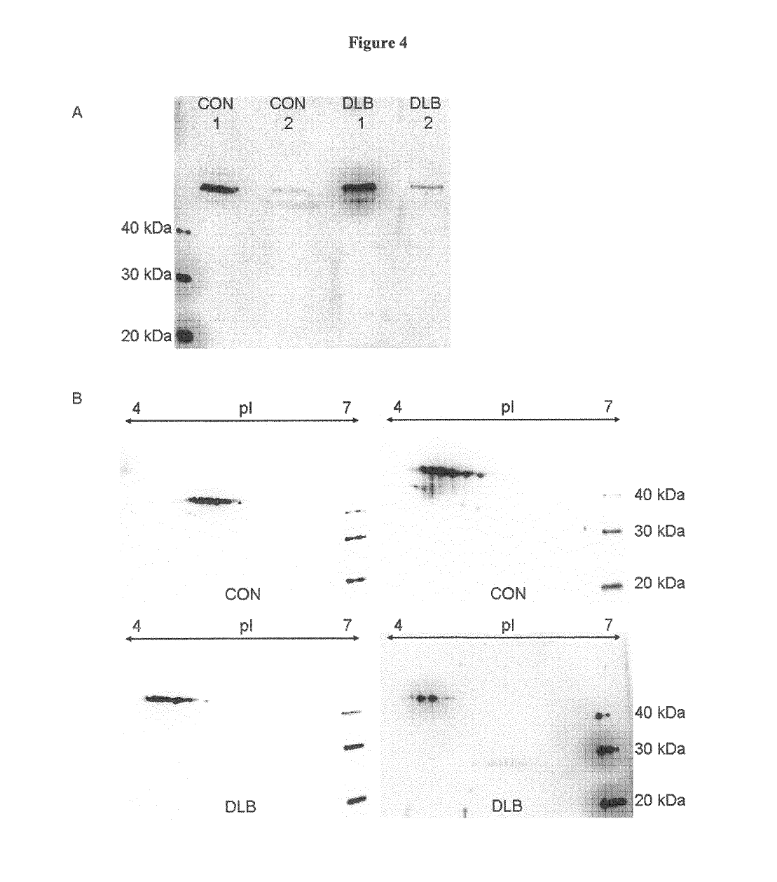

To investigate whether Serpin A1 in CSF is indeed brain derived, its expression in human cortex tissues was analyzed. 1D- and 2D-Westernimmunoblot analysis revealed Serpin A1 expression in brain material from both CON persons and patients with Lewy-body-dementia (DLB) which represent a pathologic pendant for Parkinson's dementia (PDD) (FIG. 4A). However, the additional isoforms of Serpin A1 were not restricted to patients with DLB and can also be identified in control patients (CON) (see FIG. 4B).

To investigate if the additional Serpin A1 spots were a direct result of cell destruction in the brain, tau-values above 450 pg/ml and the number of Serpin A1 spots>5/5.5 (ROC-analysis) were correlated by performing Spearman-rank correlations. No (significant) correlation was found in the various subgroups (PD: r=−0.102, p=0.663; PDD: r=0.428, p=0.0584), so that it can be concluded that the Serpin A1 isoform-distribution is a tau-level independent marker for PDD.

We claim:

1. A method for diagnosing and/or prognosing Parkinson's disease dementia (PDD) or for differential diagnosing and/or prognosing between Parkinson's disease (PD) and Parkinson's disease dementia (PDD) comprising the steps of:
(i) detecting O-glycosylation in a protein comprising a Ser/Thr motive and/or the level of sialic acid on a protein comprising a Ser/Thr motive in a biological sample from a subject, and
(ii) identifying the subject as experiencing Parkinson's disease dementia (PDD) or being prone thereto, if O-glycosylation in said protein is present or increased and/or the level of sialic acid on said protein is increased in said biological sample compared to a control.

2. The method of claim 1, wherein the biological sample is a body fluid sample.

3. The method of claim 1, wherein the subject is a human or another mammal.

4. The method of claim 1, wherein the detection of O-glycosylation comprises the step of:
(i) determining the number of glycosylated isoforms.

5. The method of claim 1, wherein the O-glycosylation is detected with an immunoassay, a gel electrophoresis, spectrometry or chromatography, or a combination thereof.

6. The method of claim 1, wherein the level of sialic acid is determined with an immunoassay, a gel electrophoresis, spectrometry or chromatography, or a combination thereof.

7. The method of claim 5, wherein
(i) the immunoassay is an enzyme immunoassay,
(ii) the gel electrophoresis is 1D or 2D gel electrophoresis,
(iii) the spectrometry is mass spectrometry (MS),
(iv) the chromatography is liquid chromatography (LC) or affinity chromatography,
(v) the chromatography is combined with spectrometry, or
(vi) the gel electrophoresis is combined with an immunoassay.

8. The method of claim 7, wherein the mass spectrometry is an electrospray ionization mass spectrometry (ESI-MS), a matrix-assisted laser desorption/ionization mass spectrometry (MALDI-MS), or an electron capture dissociation mass spectrometry (ECD-MS).

9. The method of claim 7, wherein the mass spectrometry employs tandem mass tags (TMT), isobaric tags for relative and absolute quantitation (iTRAQ), or isotope-coded affinity tags (ICATs).

10. The method of claim 1, wherein the control is
(i) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a healthy subject,
(ii) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a subject experiencing Parkinson's disease dementia (PDD), and/or
(iii) the number of O-linked glycomoieties comprised in said Ser/Thr motive comprising protein and/or the number of glycosylated isoforms of said Ser/Thr motive comprising protein, and/or the level of sialic acid on said Ser/Thr motive comprising protein known to be present in a subject experiencing Parkinson's disease (PD).

11. The method of claim 1, wherein the protein comprising a Ser/Thr motive is selected from the group consisting of a Serpin; Fetuin A; Ceruloplasmin; Haptoglobin; and Zinc-alpha-2 glycoprotein.

12. A molecule for detecting O-linked glycomoieties in a protein comprising a Ser/Thr motive and/or glycosylated isoforms of a Ser/Thr motive comprising protein for use in the diagnosis and/or prognosis of Parkinson's disease dementia (PDD) or differential diagnosis and/or prognosis between Parkinson's disease (PD) and Parkinson's disease dementia (PDD).

13. The molecule of claim 12, wherein the molecule binds to the amino acid sequence of a protein comprising a Ser/Thr motive and/or to the sialic acid on a protein comprising a Ser/Thr motive.

14. The molecule of claim 13, wherein
(i) the sialic acid binding molecule is a sialic acid specific antibody or a fragment thereof, a synthetic polypeptide, a recombinant polypeptide, a lectin, or a small molecule, or
(ii) the amino acid sequence binding molecule is a protein comprising a Ser/Thr motive specific antibody or a fragment thereof, a synthetic polypeptide, a recombinant polypeptide, a lectin, or a small molecule.

15. A means for the diagnosis and/or prognosis of Parkinson's disease dementia (PDD) or for differential diagnosis and/or prognosis between Parkinson's disease (PD) and Parkinson's disease dementia (PDD) comprising at least one molecule according to claim 12.

16. The means of claim 15, wherein said means comprises
(i) a biochip, or
(ii) a set of beads.

17. A kit for the diagnosis and/or prognosis of Parkinson's disease dementia (PDD) or for differential diagnosis and/or prognosis between Parkinson's disease (PD) and Parkinson's disease dementia (PDD) comprising
(i) a means for detecting O-glycosylation in a protein comprising a Ser/Thr motive and/or the level of sialic acid on said protein, and optionally
(ii) a data carrier, and/or
(iii) a container.

18. The kit of claim 17, wherein said data carrier comprises instructions for a method for diagnosing and/or prognosing Parkinson's disease dementia (PDD) or for differential diagnosing and/or prognosing between Parkinson's disease (PD) and Parkinson's disease dementia (PDD) comprising the steps of:
(i) detecting O-glycosylation in a protein comprising a Ser/Thr motive and/or the level of sialic acid on a protein comprising a Ser/Thr motive in a biological sample from a subject, and
(ii) identifying the subject as experiencing Parkinson's disease dementia (PDD) or being prone thereto, if O-glycosylation in said protein is present or increased and/or the level of sialic acid on said protein is increased in said biological sample compared to a control.

19-20. (canceled)
